(19) United States
(12) Patent Application Publication
(10) Pub. No.: US 2026/0335144 A1
(43) Pub. Date: Oct. 29, 2026

(54) WOUND DRESSING FOR PROVIDING PROTECTION AND ANTIMICROBIAL PROPERTIES TO A SURGICAL SITE WITH ONE OR MORE PERCUTANEOUS PINS OR WIRES

(71) Applicant: OrthoBarrier Technologies LLC, Denver, CO (US)

(72) Inventor: Andrew MAERTENS, Denver, CO (US)

(21) Appl. No.: 19/688,418

(22) Filed: May 26, 2026

Related U.S. Application Data
(63) Continuation-in-part of application No. 19/242,573, filed on Jun. 18, 2025, now Pat. No. 12,636,198.
(60) Provisional application No. 63/665,125, filed on Jun. 27, 2024.

(51) Int. Cl. A61F 13/0246; A61B 17/84; A61F 13/00

(52) U.S. Cl. CPC A61F 13/0246; A61B 17/848; A61F 13/00059; A61F 13/00063

(57) ABSTRACT

The present disclosure relates to a surgical dressing for a surgical site having a plurality of percutaneous pins or wires protruding through skin includes a unitary dressing body formed as a single continuous structure, an adhesive layer configured to attach the unitary dressing body to tissue at the surgical site, an absorbent region, an antimicrobial region, and a plurality of apertures defined by the unitary dressing body and configured to receive the plurality of percutaneous pins or wires. The unitary dressing body is configured for placement over the surgical site without assembly from separate dressing components. In use, a backing layer is removed from the adhesive layer, apertures that align with the plurality of percutaneous pins or wires are selected, and the unitary dressing body is adhered to tissue at the surgical site.

### CROSS REFERENCED TO RELATED APPLICATION

This application is a continuation-in-part of U.S. patent application Ser. No. 19/242573, filed Jun. 18, 2025, which claims the benefit of U.S. Provisional Application No. 63/665,125, filed Jun. 27, 2024, both of which are hereby incorporated by reference in their entireties as if fully set forth herein.

### BACKGROUND

### Related Technology

In orthopedic surgery, fractures that cannot be securely immobilized with a cast often require the use of percutaneous fixation devices such as Kirschner wires (K-wires), Steinmann pins, or external fixation pins configured to maintain bone alignment. During surgery, surgical pins or wires are inserted through the skin into bone following fracture reduction and are left in place for either a temporary (hours or days) or an extended period of time (weeks to months) to promote osseous stability and eventual union. The external or protruding portion of percutaneous pins and wires facilitate their eventual removal but also penetrate through the skin, creating a direct pathway for microbial entry and increasing the risk of pin site infections (PSIs).

In many situations, percutaneous pins and/or wires will protrude from the skin after they are inserted and used to hold a fractured bone together. This facilitates their removal once the bone has healed sufficiently. Accordingly, the percutaneous pins and/or wires are often left in place for extended periods (e.g., 2 weeks to 4 months). This skin penetration and external nature of the construct creates a serious risk of infection, which necessitates effective wound care to prevent infections.

FIG. 1 schematically illustrates a surgical site 100 comprising a skin surface 102, a percutaneous surgical pin/wire 104 extending outwardly from the skin 102, a wound 106, and wound exudate 108 (e.g., blood, blood components, plasma, serum, and/or serosanguineous discharges). As illustrated, the percutaneous surgical pin/wire 104 is bent to form a Shepard's crook in order to limit the protrusion distance from the skin and prevent the pin or wire from migrating under the skin surface.

FIG. 2 illustrates an example of a surgical site 200 of a badly damaged finger that has been surgically repaired using a plurality of percutaneous surgical pins or wires 204 that protrude from the skin 202 and are interconnected with a cage-like structure, a plurality of wounds 206, and wound exudate 208. The wounds 206 around the surgical pins or wires 204 are unprotected and a direct pathway for microbes to enter and infect the surgical site. Moreover, the surgical pins or wires 204 are interconnected, which complicates placement of protective bandages.

Current wound care solutions can be ad hoc and are often inadequate in preventing infection and managing wound exudate. In particular, percutaneous pins and/or wires impede proper placement of traditional wound bandages or dressings known in the art. Instead, traditional bandages must be modified by the surgeon or other medical practitioner, combined, and/or secured with external adhesive means, such as medical tape or glue, in order to protect and/or disinfect the surgical site around the protruding percutaneous pins and/or wires. Furthermore, medical practitioners and/or patients attempting to apply modified wound bandages must do so without disturbing the percutaneous pins and/or wires. Also, because traditional wound bandages are not designed for use with surgical pins, they may not reliably remain in place and/or may not provide adequate protection of the surgical site. In addition, modification of existing bandages may take prolonged time, produce errors, and make the process of protecting the surgical site more difficult and less reliable. This can be particularly problematic if the patient wishes to remove old or dirty wound dressings and replace them with fresh ones on a regular basis without proper training.

FIG. 3, for example, illustrates an example of a surgical site 300 of a foot (lower extremity) that has been surgically repaired using a plurality of percutaneous surgical pins 304 that protrude from the skin 302 and are attached to an external stabilization structure (also known as external fixator) 305, which can complicate placement of bandages. The wounds around the surgical pins 304 have been covered with makeshift bandages using typical gauze wrap and a petroleum gauze 310. One will appreciate the challenge presented to a medical practitioner when trying to provide wound care to such a surgical site. For instance, FIG. 3 illustrates the cumbersome nature of wrapping each wound and corresponding surgical pin 304 with gauze 310 in the presence of the external fixator device. Because the gauze 310 needs to be changed periodically, such difficulties will persist until the surgical pins 304 are removed and may be particularly difficult where the patient is required to change the makeshift gauze bandages on their own at home.

Commercially available dressings—such as antimicrobial foams containing agents like chlorhexidine gluconate—offer some benefit but are not specifically engineered for use at orthopedic surgical pin or wire sites. Many such products lack an adhesive layer and/or the size and structural features to facilitate secure and reliable circumferential placement around a pin or wire, thus limiting their effectiveness. Multi-step dressing systems requiring separate application of gauze, foam, followed by securement via adhesive or tape layers do not appropriately accommodate a vertically oriented pin or wire. They also introduce complexity, potential contamination, and longer exposure of the site during dressing changes. Again, this can be particularly problematic if the patient wishes to remove old or dirty wound dressings and replace them with fresh ones.

In addition, percutaneous pins and wires are themselves susceptible to microbial colonization along the external portion of the pin shaft extending above the skin. Microorganisms may adhere to the pin shaft surface and proliferate to form a “biofilm”, which are structurally and physiologically distinct from planktonic microbial populations. These biofilms exhibit a known resistance to systemic and topical antimicrobial agents. Biofilm colonization of the pin shaft can serve as a persistent reservoir of microorganisms in proximity to the pin-skin interface, contributing to recurrent or refractory pin site infection and complicating clinical management. Conventional pin site dressings and ad hoc gauze wraps deliver antimicrobial activity primarily at the skin surface and do not maintain sustained antimicrobial contact along the external pin shaft. Existing skin-surface dressings may not maintain sustained antimicrobial contact along the external shaft surface of the percutaneous pin or wire and, as a result, may leave pin-shaft biofilm insufficiently addressed.

Therefore, there is a need for a purpose-built, integrated wound dressing system that is specifically tailored to the unique requirements of orthopedic and surgical pin or wire sites.

### SUMMARY OF SOME EMBODIMENTS

Some embodiments disclosed herein address the foregoing challenges by providing a protective adhesive dressing or bandage (collectively “bandage”) that can include an adhesive and antimicrobial absorbent pad and one or more integrated slits or apertures to facilitate placement around one or more percutaneous pins or wires vertically protruding from the skin at a surgical site. In some embodiments, the disclosed bandage combines antimicrobial activity, absorbent properties, and secure adhesion in a single unit, with one or more slits that facilitate application around one or more exposed pins or wires without disturbing the implant or surgical site. This configuration can, in some embodiments, facilitate quick and secure placement around percutaneous pins or wires, while ensuring comprehensive protection, enhanced ease of use, and reduced risk of infection. Some embodiments disclosed herein are particularly beneficial as a time saving means for surgeons and medical practitioners, and for patients when it is desired to remove old or dirty wound bandages and replace them with fresh ones.

Accordingly, some embodiments disclosed herein satisfy the need for wound bandages that are specifically engineered to provide antimicrobial protection both to the patient and the pin itself, absorb exudate, and promote healing around percutaneous pins and wires at a surgical site. Some embodiments disclosed herein provide novel, yet simple to use, wound care bandages designed specifically for percutaneous pins and/or wires. Such bandages may comprise a layered structure that provides the combination of antimicrobial properties, exudate absorption, and mechanical stability (i.e., through adhesion). In some embodiments, the bandage includes one or more slits that pass partially across the width or diameter of the bandage to facilitate circumferential placement around a protruding pin or wire and an adhesive border or layer to secure the bandage in place at the surgical site. In some embodiments, the slits can be partial thickness slits. In some embodiments, the slits can extend across an entire width or diameter of the bandage to facilitate circumferential placement around a protruding pin or wire and an adhesive border or layer to secure the bandage in place at the surgical site. Some embodiments of the disclosed surgical dressings are designed to reduce the risk of infection, improve patient comfort and compliance, facilitate easier wound monitoring and care, and facilitate removal and replacement of old or dirty bandages with fresh bandages, including by healthcare practitioners and patients.

Disclosed herein are embodiments a surgical dressing for a surgical site having a plurality of percutaneous pins or wires protruding through skin. In some embodiments, the surgical dressing can include a unitary dressing body, and an adhesive layer configured to attach the unitary dressing body to tissue at the surgical site, an absorbent region, and a plurality of tearable perforated regions configured to selectively be torn to form a plurality of apertures, the plurality of apertures configured to receive the plurality of percutaneous pins or wires, wherein the surgical dressing is configured for placement over the surgical site to prevent microbial infection of the surgical site. In some embodiments, the surgical dressing can also have an antimicrobial region.

Any embodiments of the devices, systems, and methods disclosed herein can include, in additional embodiments, one or more or any combination of the following features, components, and/or details, in any combination with any of the other features, components, and/or details of any other embodiments disclosed herein: wherein at least one of the plurality of perforated regions includes a perforation pattern including a slit, a circle, a keyhole shape, and/or a multi-segment compliant opening; wherein one or more of the plurality of perforations are arranged in a pre-defined grid pattern; wherein the pre-defined grid pattern is a square grid pattern, a hexagonal grid pattern, a pentagonal grid pattern, or a radial pattern; wherein the one or more perforations include perforations separated by intervening land portions having a width of 0.3 mm to 2 mm; further including an adhesive material disposed along an interior surface of at least one of the plurality of apertures; wherein the surgical dressing is configured to attenuate transmission of motion from at least one of the plurality of percutaneous pins or wires to the surgical site; wherein the surgical dressing further includes one or more scores extending partially through one or more layers of the unitary dressing body; wherein the antimicrobial region and the absorbent region include discrete layers adhered to one another within the unitary dressing body; wherein the antimicrobial region and the absorbent region are at least partially overlapping or substantially coextensive within the unitary dressing body; and/or wherein the antimicrobial region has a greater concentration of antimicrobial agent near a respective aperture than in a region spaced farther from the aperture;

Any embodiments of the devices, systems, and methods disclosed herein can include, in additional embodiments, one or more or any combination of the following features, components, and/or details, in any combination with any of the other features, components, and/or details of any other embodiments disclosed herein: wherein the unitary dressing body includes a visual indicator configured to provide a visible indication that the surgical dressing is to be replaced; wherein the visual indicator is configured to provide the visible indication by changing color; wherein the visual indicator is configured to provide the visible indication by changing in opacity; wherein the visual indicator is configured to provide the visible indication in response to time in use, a depletion of an antimicrobial agent in the antimicrobial material, or an accumulation of exudate; wherein the visual indicator is configured to provide the visible indication in response to a level of contamination exceeding a predetermined value; wherein the visual indicator is configured to provide the visible indication in response to time in use; wherein the visual indicator is configured to provide the visible indication when a pH exceeds about 8.0; wherein the visual indicator is configured to provide the visible indication when a moisture level exceeds about 50-75%; further including a supporting backing layer disposed over a surface of the bandage opposite the skin-contacting surface; wherein the antimicrobial region includes an antimicrobial agent; wherein the antimicrobial agent is polyhexamethylene biguanide or chlorhexidine gluconate; wherein the antimicrobial agent is included in a weight-per-weight concentration of antimicrobial agent to antimicrobial region of about 0.1% to about 2.0%; wherein the unitary dressing body includes a visual indicator configured to provide a visible indication when a concentration of an antimicrobial agent is below about 0.1% to about 2.0%; further including a supporting backing layer disposed over a surface of the bandage opposite the skin-contacting surface; wherein the antimicrobial region includes polyurethane foam; wherein the adhesive layer extends across substantially all of a skin-contacting surface of the surgical dressing; wherein the aperture diameter is about 3 mm to about 6 mm; wherein the aperture diameter is about 2 mm to about 5 mm; wherein the thickness of the surgical dressing is about 15 mm to about 25 mm; wherein the diameter of the surgical dressing is about 45 mm to about 55 mm; and/or wherein the absorbent region includes a first absorbent material positioned closer to a respective percutaneous pin or wire and a second absorbent material positioned farther from the respective percutaneous pin or wire, the second absorbent material having a greater absorbency than the first absorbent material to promote transfer of moisture or exudate away from the respective percutaneous pin or wire.

In some embodiments of the bandage for providing wound care at a surgical site with a percutaneous pin or wire, the bandage comprises an adhesive layer, an absorbent layer, an antimicrobial material forming a layer (e.g., without limitation, between the adhesive and absorbent layers) and/or that is impregnated in the adhesive layer and/or the absorbent layer, and a slit configured to facilitate placement of the bandage around a percutaneous pin or wire. The slit may lead to or terminate at a central opening, which may be selected to correspond to the diameter of the percutaneous pin or wire to ensure good circumferential contact of the absorbent material of the layer around the pin or wire. in some embodiments, a first side of the adhesive layer can be configured to adhere to tissue, and a second side opposite the first side is configured for adhesion of the absorbent and/or antimicrobial layer. The adhesive layer may comprise, as nonlimiting examples, a hydrocolloid, acrylic polymer film, cyanoacrylate film, silicone film, silicone, and/or other material capable of attaching to skin at a surgical site. The bandage further includes a backing layer adjacent to the first side of the adhesive layer to protect the adhesive (such as to prevent contamination and/or self-adhesion) prior to use. In some embodiments, the adhesive layer facilitates attachment of the bandage to tissue and/or the percutaneous pin or wire at the surgical site without the use of tape.

In some embodiments, the bandage may further include an absorbent layer adjacent (e.g., adhered) to the second side of the adhesive layer. In some embodiments, the absorbent layer is configured to absorb fluids (e.g., blood, plasma, serum, and/or serosanguineous discharges that may leak from the surgical site). The absorbent layer may advantageously comprise a porous and/or hydrophilic material formulated and be configured to absorb fluids (exudate) discharged from the surgical site, including at the interface of patient tissue and the percutaneous pin or wire. Non-limiting examples include silicone, cotton wool, gauze, lint, plasters, foam, polyurethane foam, alginate, hydrocolloid, hydrogel, and/or others capable of absorbing fluid leakage from a surgical or other wound site.

In some embodiments, the absorbent layer can include a first absorbent material having a first absorbency and a second absorbent material having a second absorbency greater than the first absorbency. The first absorbent material may be positioned closer to the percutaneous pin or wire, and the second absorbent material may be positioned farther from the percutaneous pin or wire. In some embodiments, the second absorbent material comprises a superabsorbent polymer. This arrangement can promote transfer of absorbed moisture or exudate from the first absorbent material toward the second absorbent material, thereby directing fluid away from the percutaneous pin or wire and reducing pooling of fluid at the pin or wire.

In some embodiments, the absorbent layer may optionally be impregnated with a hemostatic agent. In some embodiments, the hemostatic agent may comprise one or more of thrombin, thrombin enzyme, prothrombin complex concentrate, dried plasma, cyanoacrylate, fibrin sealant, tranexamic acid, microfibrillar collagen, microporous polysaccharide spheres, gelatin matrix, oxidized regenerated cellulose, alginate, albumin, glutaraldehyde, alum (hydrated salts of aluminum and alkali metals) aluminum chloride, zinc chloride, and ferric sulfates.

In some embodiments, the bandage may further include an antimicrobial agent impregnated in or positioned adjacent to at least one of the adhesive layer or the absorbent layer. In some embodiments, the antimicrobial agent in the antimicrobial layer and/or impregnated in the absorbent layer and/or adhesive layer, is configured to provide antimicrobial protection at the surgical site. The term “provide antimicrobial protection” can broadly mean any antiseptic activity in which microbes and biofilms are killed, destroyed, and/or prevented from forming at and/or proliferating and infecting the surgical site. In some embodiments, the antimicrobial agent may include, but is not limited to, one or more of silver (e.g., silver nanoparticles and silver salts, such as silver nitrate and silver sulfadiazine, ionic silver, and nanocrystalline silver), iodine (e.g., povidone iodine and cadexomer iodine), chlorhexidine (e.g., chlorhexidine gluconate), polyhexamethylene biguanide (PHMB), honey, antibiotics (e.g., gentamicin, mupirocin, and bacitracin), hypochlorous acid, gentian violet, and essential oils (e.g., tea tree oil).

In some embodiments, the bandage further includes a supportive backing layer disposed over a surface of the bandage opposite the skin-contacting surface. The supportive backing layer may overlie one or more other layers of the bandage and may provide structural support, barrier protection, handling stability, or protection of the underlying layers before or during use. In some embodiments, the supporting backing layer is removably coupled to the bandage and is removed before application. In other embodiments, the supporting backing layer remains coupled to the bandage after application to provide a protective outer surface. In some embodiments, the supporting backing layer is made of polyurethane foam.

In some embodiments, the bandage can further include a slit passing partially across the width or diameter of the bandage, preferably through each layer of the bandage. The slit is configured to facilitate circumferential placement of the bandage around and accommodate a percutaneous pin or wire passing through the bandage in a desired position. In some embodiments, the slit may pass approximately halfway across the diameter or width of the bandage, although it can be more or less than halfway as desired to accommodate a surgical site. The absorbent layer may be configured to circumferentially contact and close around the pin or wire to provide an antimicrobial barrier and absorb exudates from the wound. In some embodiments, an interior surface of the slit is lined with an adhesive material so that, after the bandage is positioned around the pin at the surgical site, opposing portions of the slit can be secured together to close the bandage around the pin.

In some embodiments, the bandage can have a desired geometric shape. For example, the bandage may be circular, rectangular, triangular, pentagonal, hexagonal, octagonal, or provided in a sheet. The geometric shape of the bandage may facilitate the placement of a plurality of bandages at a surgical site that has more than one exposed percutaneous pin or wire. In some implementations, a plurality of bandages having advantageous geometric shapes can be arranged to form an uninterrupted barrier at the surgical site. In some embodiments, the bandage is provided as a sheet of hexagonally tessellated individual regions separated by perforations.

As used herein for some embodiments, the term “unitary” can refer to a dressing body that is provided as a single integrated body or structure before application to a surgical site, including embodiments in which the dressing body includes perforations, score lines, tearable regions, removable portions, perforated regions defining potential apertures, or other modification features configured to permit adjustment of the shape or aperture arrangement of the dressing body.

In some embodiments, a bandage comprises a unitary multi-aperture dressing body configured for placement over a surgical site having multiple percutaneous pins or wires. The dressing body can include one or more or a plurality of pre-formed apertures positioned in a predetermined pattern corresponding to common external fixator geometries, such that a practitioner can select which apertures more closely align with the percutaneous pins during application while avoiding assembly of separate dressing components in abutment.

For example, in some embodiments intended for application to a finger or a hand, the space between apertures may be about 5 mm to about 100 mm, or about 5 mm to about 10 mm, or about 5 mm to about 20 mm, or about 5 mm to about 7 mm, or about 10 mm to about 15 mm, or 5 mm to 100 mm, or 5 mm to 10 mm, or 5 mm to 20 mm, or 5 mm to 7 mm, or 10 mm to 15 mm, or of any values, approximate values, ranges of values, or approximate ranges of values in any of the foregoing ranges.

In some embodiments intended for application to a finger or a hand, the apertures may have a diameter of about 1 mm to about 3 mm, or about 2 mm to about 3 mm, or about 2 mm to about 4 mm, or about 2 mm to about 5 mm, or 1 mm to 3 mm, or 2 mm to 3 mm, or 2 mm to 4 mm, or 2 mm to 5 mm, or of any values, approximate values, ranges of values, or approximate ranges of values in any of the foregoing ranges.

In some embodiments intended for application to a finger or a hand, the dressing body may have an overall thickness of about 15 mm to about 25 mm, or 17 mm to about 23 mm, or about 18 mm to about 22 mm, or about 19 mm to about 21 mm, or 15 mm to 25 mm, or 17 mm to 23 mm, or 18 mm to 22 mm, or 19 mm to 21 mm, or of any values, approximate values, ranges of values, or approximate ranges of values in any of the foregoing ranges.

In some embodiments intended for application to a finger or a hand, individually tessellated regions of the dressing body may be hexagonal in shape.

For example, in some embodiments intended for application to a wrist, a forearm, or a lower extremity, the space between apertures may be about 0.5 cm to about 30 cm, or about 5 cm to about 10 cm, or about 1 cm to about 2 cm, or about 5 cm to about 15 cm, or 0.5 cm to 30 cm, or 5 cm to 10 cm, or 1 cm to 2 cm, or 5 cm to 15 cm, or of any values, approximate values, ranges of values, or approximate ranges of values in any of the foregoing ranges.

In some embodiments intended for application to a wrist, forearm, or lower extremity, the apertures may have a diameter of about 0.5 mm to about 5 mm, or about 0.62 mm to about 6 mm, or about 3 mm to about 5 mm, or about 4 mm to about 5 mm, or about 4 mm to about 6 mm, or about 4 mm to about 7 mm, or 0.5 mm to 5 mm, or 0.62 mm to 6 mm, or 3 mm to 5 mm, or 4 mm to 5 mm, or 4 mm to 6 mm, or 4 mm to 7 mm, or of any values, approximate values, ranges of values, or approximate ranges of values in any of the foregoing ranges.

In some embodiments intended for application to a wrist, forearm, or lower extremity, the dressing body may have an overall thickness of about 45 mm to about 55 mm, or about 47 mm to about 53 mm, or about 48 mm to about 52 mm, or about 49 mm to about 51 mm, or 45 mm to 55 mm, or 47 mm to 53 mm, or 48 mm to 52 mm, or 49 mm to 51 mm, or of any values, approximate values, ranges of values, or approximate ranges of values in any of the foregoing ranges.

In some embodiments intended for application to a wrist, forearm, or lower extremity, individually tessellated regions of the dressing body may be hexagonal in shape.

For example, in some embodiments intended for application to an external fixator construct of a tibia, pilon, or other long bone, the space between apertures may be about 47 mm to about 53 mm, or about 48 mm to about 52 mm, or about 49 mm to about 51 mm, or 47 mm to 53 mm, or 48 mm to 52 mm, or 49 mm to 51 mm, or of any values, approximate values, ranges of values, or approximate ranges of values in any of the foregoing ranges.

In some embodiments intended for application to an external fixator construct of a tibia, pilon, or other long bone, the apertures may have a diameter of about 3 mm to about 6 mm, or about 4 mm to about 5 mm, or about 4 mm to about 6 mm, or about 4 mm to about 7 mm, or 3 mm to 6 mm, or 4 mm to 5 mm, or 4 mm to 6 mm, or 4 mm to 7 mm, or of any values, approximate values, ranges of values, or approximate ranges of values in any of the foregoing ranges.

In some embodiments intended for application to an external fixator construct of a tibia, pilon, or other long bone, the dressing body may have an overall thickness of about 40 mm to about 50 mm, or about 47 mm to about 53 mm, or about 48 mm to about 52 mm, or about 49 mm to about 51 mm, or 40 mm to 50 mm, or 47 mm to 53 mm, or 48 mm to 52 mm, or 49 mm to 51 mm, or of any values, approximate values, ranges of values, or approximate ranges of values in any of the foregoing ranges.

In some embodiments intended for application to an external fixator construct of a tibia, pilon, or other long bone, individually tessellated regions of the dressing body may be hexagonal in shape.

In some embodiments, the apertures may be formed during manufacture of the dressing body by die cutting, laser cutting, water-jet cutting, mechanical punching, molding, or another material-removal or material-forming process selected based on the composition and thickness of the dressing layers. In some embodiments, the apertures and any associated slits, perforation lines, or tear-initiation features are created in a single manufacturing step or in sequential converting steps to provide consistent aperture geometry, spacing, and edge quality across the unitary multi-aperture dressing.

In some embodiments, a clinician may create one or more apertures at desired locations at the time of use based on the observed positions of the percutaneous pins or wires. The apertures may be made manually using scissors, a hole-punching tool, or another cutting instrument, thereby allowing the dressing to be customized intraoperatively or at the point of care to accommodate patient-specific pin spacing and external fixator geometry. The apertures may also be made manually by manipulating the material to tear along perforated or pre-cut regions defining potential apertures, or by tearing along perforated or pre-cut regions to remove plugs of material.

In some embodiments, the dressing body may include one or more scored regions formed by a cutting tool to locally reduce bending stiffness and promote flexibility of the dressing body during placement around a plurality of pin sites or during storage and transport of the dressing body. The scored regions may extend partially through one or more layers of the dressing without fully penetrating the dressing body, and may be arranged as linear scores, curved scores, intersecting scores, discontinuous scores, or patterned score arrays positioned between apertures, adjacent apertures, along peripheral regions, or in other selected portions of the dressing to facilitate conformance to patient anatomy and external fixator geometry while maintaining overall structural integrity and to facilitate ease of folding during storage and transport of the dressing body.

In some embodiments, the dressing body is provided as a sheet-shaped unitary structure and is packaged in a vacuum-sealed condition to reduce package volume, maintain a sterile barrier until use, and facilitate storage and transport within clinical supply chains. One or more scored regions may be formed in the dressing body such that the sheet-shaped structure can be folded, flexed, or compactly arranged for storage and transport and can subsequently be unfolded or otherwise deployed for application at a treatment site, with the scored regions promoting controlled bending while reducing a likelihood of unintended tearing or deformation of surrounding portions of the dressing body.

In some embodiments, the unitary multi-aperture dressing body comprises one or more antimicrobial regions positioned to interface with one or more percutaneous pins extending through the dressing body and one or more absorbent regions positioned to interface with one or more percutaneous pins extending through the dressing body. The antimicrobial region comprises a suitable antimicrobial agent, which may include, by way of non-limiting example, chlorhexidine gluconate (CHG), chlorhexidine salts, silver, silver ions, silver sulfadiazine, silver oxide, ionic silver compounds, iodine, povidone-iodine, polyhexamethylene biguanide (PHMB), octenidine, benzalkonium chloride, benzethonium chloride, triclosan, copper, copper salts, zinc, zinc salts, antibiotics, antiseptics, antimicrobial peptides, antifungal agents, antiviral agents, and combinations thereof. In some embodiments, the antimicrobial agent is incorporated into, coated onto, impregnated within, bonded to, infused into, or otherwise associated with a foam, polyurethane foam, fabric, polymeric matrix, hydrogel, film, absorbent layer, or other portion of the dressing body. In some embodiments, the antimicrobial agent is PHMB and is provided in a weight-per-weight concentration of antimicrobial agent to foam of about 0.1% to about 2.0%, or about 1.0% to about 2.0%, or about 0.5% to about 2.0%, or about 0.1% to about 1.0%, or about 0.1% to about 0.5%, or 0.1% to 2.0%, or 1.0% to 2.0%, or 0.5% to 2.0%, or 0.1% to 1.0%, or 0.1% to 0.5%, or of any values, approximate values, ranges of values, or approximate ranges of values in any of the foregoing ranges.

In some embodiments, the one or more antimicrobial regions are initially provided as a single sheet of antimicrobial material before formation of the one or more apertures, and the one or more absorbent regions are initially provided as a single sheet of absorbent material before formation of the one or more apertures. The single sheet of antimicrobial material and the single sheet of absorbent material are assembled in a layered arrangement and bonded to one another by chemical bonding, thermal bonding, adhesive bonding, ultrasonic bonding, mechanical bonding, and/or another suitable attachment technique such that the bonded sheets collectively define the unitary dressing body. Thereafter, in some embodiments, the one or more apertures can be formed through the unitary dressing body such that each aperture extends through the single sheet of antimicrobial material and through the single sheet of absorbent material. As a result, interior surfaces of each aperture expose cross-sectional portions of both the antimicrobial material and the absorbent material such that both materials may directly interface with the pin received within the aperture.

In some embodiments, an antimicrobial region is configured as a coaxially shaped region positioned about the pin The coaxially shaped region may comprise an elongate antimicrobial interface surrounding at least a portion of the pin external to the skin, thereby maintaining antimicrobial contact along the length of the pin and reducing microbial presence in a region extending outwardly from the pin-site opening. In some preferred embodiments, the antimicrobial region is configured to maximize contact with the outer surface of the pin along the portion of the pin engaged by the coaxially shaped region. In some embodiments, the unitary dressing body includes a plurality of antimicrobial regions configured as a coaxially shaped region. In some embodiments, the antimicrobial coaxially shaped region functions as a sustained antimicrobial reservoir positioned coaxially about the external surface of the percutaneous pin or wire.

In some embodiments, the coaxially shaped region may be configured to maintain antimicrobial contact along the external pin shaft over an extended duration of use. In some embodiments, the antimicrobial coaxially shaped region can prevent biofilm formation on the external surface of the pin shaft by way of sustained antimicrobial contact along the external pin shaft, thereby reducing the establishment of pin-shaft microbial reservoirs that can contribute to pin site infection.

In some embodiments, the antimicrobial region combines the single-sheet configuration and coaxially shaped configuration such that the antimicrobial material is initially provided as a unitary sheet having one or more coaxially shaped regions formed integrally with, and projecting from, the plane of the sheet at locations corresponding to the one or more apertures to be formed in the unitary dressing body. The one or more coaxially shaped regions may extend substantially transverse to the plane of the antimicrobial sheet so that, after the antimicrobial sheet is assembled with the absorbent sheet, each coaxially shaped region is aligned with and surrounds a respective aperture to receive a percutaneous pin therethrough. In some embodiments, the apertures may be pre-formed in the absorbent sheet prior to assembly of the absorbent sheet with the antimicrobial sheet, such that after assembly the coaxially shaped regions extend substantially through the pre-formed apertures in the absorbent sheet. Thereafter, in some embodiments, apertures may be then formed in the coaxially shaped regions of the antimicrobial region such that the apertures extend through substantially all of the antimicrobial sheet and the absorbent sheet. In this manner, the planar portion of the antimicrobial sheet may provide antimicrobial coverage within the dressing body while the coaxially shaped regions provide an elongate antimicrobial interface extending coaxially along the pin from the skin surface toward the distal end of the pin. In some embodiments, the coaxially shaped regions are pre-formed before bonding of the antimicrobial sheet to the absorbent sheet, and the bonded structure collectively defines the unitary multi-aperture dressing body.

In a further embodiment, the sheet portion of the combined antimicrobial region is positioned such that, during use, the sheet portion is spaced from and does not directly contact the patient's skin, thereby reducing or avoiding exposure of healthy skin to the antimicrobial material. In such an arrangement, the antimicrobial functionality may be concentrated at the coaxially shaped regions associated with the one or more apertures so that antimicrobial contact is provided primarily along the pin interfaces while limiting irritation, sensitivity, or other undesirable effects that could result from antimicrobial contact with surrounding healthy tissue.

In a further embodiment, the combined antimicrobial region comprises a planar region and one or more coaxially shaped regions, wherein the planar region is positioned such that it does not directly contact the patient's skin during use. The planar region may comprise a relatively faster-release antimicrobial-impregnated material configured to function as a reservoir of antimicrobial agent, while the one or more coaxially shaped regions comprise a relatively slower-release antimicrobial-impregnated material positioned to surround and directly interface with the pin. In this manner, the planar region may supply or replenish antimicrobial agent to the one or more coaxially shaped regions, and the one or more coaxially shaped regions may provide sustained antimicrobial contact along the pin while reducing or avoiding direct antimicrobial exposure to surrounding healthy skin. Differential release behavior between the planar region and the one or more coaxially shaped regions may be achieved through selection of different antimicrobial agents, carrier materials, concentrations, coatings, porosities, material densities, layer structures, or combinations thereof. In some embodiments, the relatively slower-release antimicrobial-impregnated material of the one or more coaxially shaped regions is selected to provide a sustained antimicrobial presence along the external pin shaft sufficient to inhibit microbial colonization and biofilm formation on the exterior surface of the pin or percutaneous wire over an extended period of use. In some embodiments, the coaxially shaped region is configured to maintain antimicrobial contact over a larger surface area of the external pin shaft than an antimicrobial agent applied only at the skin surface. The coaxially shaped region may thereby provide effective antimicrobial activity along the external pin shaft for a longer duration than an equivalent quantity of antimicrobial agent applied only at the skin surface.

In some embodiments, the antimicrobial region and the absorbent region may at least partially overlap or may be substantially coextensive within the unitary dressing body. For example, a single material may be engineered to provide both absorbent and antimicrobial functionality, such that the material is configured to wick, absorb, or otherwise manage moisture while also inhibiting microbial presence at or near the pin site. In some embodiments, the single material can have spatially varying porosity, density, antimicrobial loading, hydrophilicity, or other material characteristics selected to provide desired absorptive performance, antimicrobial performance, or a combination thereof at different regions of the dressing. In some embodiments, moisture wicking is achieved at least in part by capillary action through pores, channels, fibers, cells, interstitial spaces, or other fluid pathways within the material. The size, shape, distribution, and surface characteristics of such pathways may be selected to draw moisture away from the pin site and into adjacent portions of the dressing body, thereby promoting fluid management and a drier local interface.

In some embodiments, the combined antimicrobial absorbent material comprises a solid foam having pores sized, distributed, or otherwise selected to promote moisture wicking and fluid uptake, wherein the foam is further impregnated, coated, infused, or otherwise combined with an antimicrobial agent. In this manner, a single region or layer of the dressing body may simultaneously provide absorbent performance and antimicrobial performance while directly interfacing with one or more percutaneous pins. In some embodiments, the foam is a polyurethane foam.

In a further embodiment, the combined antimicrobial absorbent material is configured to provide a relatively slow release of antimicrobial agent at the pin-skin interface and a release profile that increases as a direct function of distance from the pin. Stated differently, portions of the material positioned closer to the pin may retain and release the antimicrobial agent more gradually, while portions positioned farther from the pin may release the antimicrobial agent more rapidly. Such a gradient release profile may be achieved by varying antimicrobial loading, material density, pore structure, coating thickness, carrier composition, crosslinking, or other material characteristics as a function of position within the dressing body. In this manner, the material may maintain sustained antimicrobial presence at the pin-skin interface while portions positioned farther from the pin may supply or replenish antimicrobial agent to the one or more regions contacting the pin-skin interface.

In some embodiments, the bandage comprises a compliant material configured to damp motion of the pin relative to surrounding tissue. The compliant material may deform in response to movement-induced forces so as to absorb, distribute, or attenuate mechanical loading at the pin site, thereby reducing localized irritation and promoting stabilization of the pin-tissue interface.

In some embodiments, the compliant material is a foam selected and engineered to have pore sizes, porosity, density, resilience, and material properties sufficient to provide damping at the pin site. In this manner, the foam may absorb and attenuate motion-induced forces so as to reduce shear stress and mechanical agitation at the wound site, thereby promoting a more stable local environment for healing.

In some embodiments, the compliant interface region is configured as a mechanical energy-dissipating interface that modulates transfer of force between the percutaneous member and adjacent tissue, rather than merely functioning as a passive cushion. In some embodiments, the compliant interface region includes an adhesive material disposed along a pin-contacting surface of the bandage. The adhesive material can help secure the bandage relative to the percutaneous member and can reduce transmission of shear, impact, or other mechanically irritating forces from the percutaneous member to tissue surrounding the percutaneous member. In some embodiments, the compliant interface region deforms in a controlled manner to absorb, redistribute, and attenuate motion-induced loading, and can reduce transmission of shear, impact, and other mechanically irritating forces to the tissue surrounding the percutaneous member. In some embodiments, the compliant interface region and the adhesive material cooperate to absorb and redistribute motion imparted by the pin or percutaneous wire within the dressing materials. By absorbing and redistributing pin-induced motion within the dressing materials, the surgical dressing can reduce shear forces and tract-disrupting motion at skin tissue immediately surrounding the percutaneous pin or wire, thereby reducing pin-tract widening and disruption of an epithelial seal at the pin-skin interface.

In some embodiments, the material properties providing compliance described herein are provided independently of any antimicrobial feature of the dressing body, such that mechanical protection or damping at the pin site may be provided without antimicrobial activity. In other embodiments, the material properties providing compliance are provided in combination with one or more antimicrobial material properties described herein, such that the dressing body provides both mechanical protection and antimicrobial protection at the pin site.

In some embodiments, the unitary dressing includes one or more perforations, distinct from the one or more apertures configured to receive percutaneous pins, to facilitate tearing, trimming, or cutting of the dressing into a desired shape for a particular patient geometry or treatment configuration. The perforations may define tear lines, cut guides, separable portions, contouring regions, or size-adjustment regions that enable a clinician to selectively modify the outer shape, perimeter, or local configuration of the dressing while preserving desired functionality at pin-receiving locations. In some embodiments, the perforations are separated by intervening land portions having, for example and without limitation, a width of about 0.3 mm to about 2 mm, or about 0.3 mm to about 0.5 mm, or about 0.3 mm to about 1 mm, or about 1 mm to about 2 mm, or 0.3 mm to 2 mm, or 0.3 mm to 0.5 mm, or 0.3 mm to 1 mm, or about 1 mm to 2 mm, or of any values, approximate values, ranges of values, or approximate ranges of values in any of the foregoing ranges. In some embodiments, the perforations are arranged to follow a hexagonal grid pattern, a pentagonal grid pattern, or another polygonal grid pattern to facilitate selective tearing, trimming, or contouring of the dressing body. In some embodiments, the unitary dressing body comprises a plurality of hexagonal tile regions tessellated such that adjacent tile regions share edges, with each tile region defining one of the plurality of apertures at or proximate its geometric center. The hexagonal tessellation can provide edge-sharing coverage of an area of the surgical site with reduced inter-tile gap area as compared to circular or square tile arrangements of comparable aperture spacing, and accommodates aperture spacing arrangements corresponding to triangular, radial, and offset multi-pin configurations commonly encountered in external fixation. In some embodiments the tile regions may be circular, square, rectangular, pentagonal, triangular, octagonal, or of another polygonal or curvilinear shape, or any combinations thereof.

In some embodiments, the unitary dressing further includes a visual indicator configured to provide a visible indication of when the dressing should be replaced. For example and without limitation, in some embodiments, the dressing can be configured to provide a visible indication that of at least a portion of the surgical dressing has reached a predetermined state. The visual indicator or other portion of the dressing may change color, opacity, pattern, or another visible characteristic in response to a predetermined condition of a portion of the dressing or the visual indicator reaching a predetermined state, such as, without limitation, time in use (e.g., 5 days or more, 3 days, 2 days, 12 hours or less, etc.), moisture exposure, exudate accumulation, antimicrobial depletion, contamination, microbial enzymatic activity, pH change, temperature change, or another replacement-related condition. In some embodiments, the change in the visible characteristic may be in response to absorption of wound exudate by the absorbent region.

In some embodiments, any of the foregoing conditions reaching or exceeding a predetermined state or value can trigger the visible indication. For example, in some embodiments, the change in the visible characteristic may indicate that the surgical dressing has reached a predetermined saturation level. In some embodiments, the visual indicator may change color when the absorbed moisture in the visual indicator is about 50% to about 75%, or about 60% to about 75%, or about 65% to about 75%, or about 65% to about 70%, or about 50% to about 60%, or 50% to 75%, or 60% to 75%, or 65% to 75%, or 65% to 70%, or 50% to 60%, or of any values, approximate values, ranges of values, or approximate ranges of values in any of the foregoing ranges. In some embodiments, the change in the visible characteristic can be in response to depletion of antimicrobial agent within the antimicrobial region, indicating that the surgical dressing has reached a predetermined level of antimicrobial depletion. In some embodiments, the visual indicator may change color when the concentration of antimicrobial agent is about 0.1% to about 2.0%, or about 0.1% to about 1.0%, or about 0.1% to about 0.5%, or about 0.1% to about 0.2%, or 0.1% to 2.0%, or 0.1% to 1.0%, or 0.1% to 0.5%, or 0.1% to 0.2%, or of any values, approximate values, ranges of values, or approximate ranges of values in any of the foregoing ranges. In some embodiments, the visual indicator can be configured to change a visible characteristic in response to a change in pH at an interface between the surgical dressing and tissue at the surgical site. In some embodiments, the visual indicator may change in color when pH exceeds 8.0 or about 8.0.

In some embodiments, the bandage for providing wound care at a surgical site with one or more percutaneous pins or wires can have an adhesive layer with a first side configured to adhere to tissue and a second side opposite the first side. The bandage can further include a backing layer adjacent to the first side of the adhesive layer and an absorbent layer adjacent to the second side. The bandage further includes an antimicrobial agent in and/or adjacent to at least one of the adhesive layer or the absorbent layer. The bandage further includes a plurality of slits passing through the bandage (i.e., passing partially across the width or diameter and through each layer of the bandage). The plurality of slits are configured to accommodate a plurality of percutaneous pins or wires received therethrough. Alternatively, the plurality of slits may simply provide a plurality of locations that can best accommodate a percutaneous pin or wire at a given location at the surgical site.

In some embodiments, the method of protecting and providing an antimicrobial barrier at a surgical site with one or more percutaneous pins and/or wires extending therefrom includes providing one or more bandages comprised of an adhesive layer, an absorbent layer, and an antimicrobial agent forming a separate layer and/or impregnated in the adhesive layer and/or absorbent layer, and one or more slits extending partially across the width or diameter of the bandage and through the adhesive layer, antimicrobial agent, and absorbent layer. The method further involves removing a backing layer from a first side of the adhesive layer and placing the adhesive layer at the surgical site such that each of the one or more percutaneous pins or wires extends through a respective slit of one or more bandages. The absorbent layer absorbs any fluids or exudate that may leak out of the surgical site, including the wound caused by the percutaneous pin or wire, and the antimicrobial agent disinfects the surgical site (e.g., maintains a sterile environment at the surgical site by providing an antimicrobial barrier).

These and other advantages and features of the invention will become more fully apparent from the following description and appended claims or may be learned by the practice of the invention as set forth hereinafter.

### BRIEF DESCRIPTION OF THE DRAWINGS

In order to describe the manner in which at least some of the advantages and features of the invention may be obtained, a more particular description of embodiments of the invention will be rendered by reference to specific embodiments thereof which are illustrated in the appended drawings. Understanding that these drawings depict only typical embodiments of the invention and are not therefore to be considered to be limiting of its scope, embodiments of the invention will be described and explained with additional specificity and detail through the use of the accompanying drawings.

FIG. 1 schematically illustrates a surgical site with a percutaneous pin or wire protruding from the skin;

FIG. 2 illustrates a surgical site with a badly crushed finger that has been surgically repaired using a plurality of percutaneous pins or wires that protrude from the skin;

FIG. 3 illustrates a surgical site with a foot that has been surgically repaired using a plurality of percutaneous pins or wires that protrude from the skin and which are covered with makeshift gauze bandages;

FIG. 4A is a top perspective view and FIG. 4B is a side cross-sectional view that schematically illustrate a first embodiment of a bandage designed to protect and provide an antimicrobial barrier at a surgical site with one or more percutaneous pins or wires protruding from the skin;

FIG. 5A is a top perspective view and FIG. 5B is a side cross-sectional view that schematically illustrate a surgical site with a percutaneous pin or wire protruding from the skin and a protective bandage according to the disclosure applied at the surgical site, with the percutaneous pin or wire passing through a slit of the bandage;

FIGS. 6A-6E schematically illustrate bandages according to the disclosure that have various geometric shapes designed to protect and provide an antimicrobial barrier at a surgical site with one or more percutaneous pins or wires protruding from the skin;

FIG. 7A is a perspective view of a surgical site with a plurality of percutaneous pins or wires protruding from the skin;

FIG. 7B is a perspective view of the surgical site of FIG. 7A with a plurality of bandages applied at the surgical site and with the percutaneous pins or wires extending through respective slits of the bandages;

FIGS. 8A and 8B schematically illustrate embodiments of a bandage having a plurality of slits designed to protect and provide an antimicrobial barrier at a surgical site with a plurality of percutaneous pins or wires protruding from the skin;

FIG. 9 illustrates two bandages with different slit designs to protect and provide an antimicrobial barrier at a surgical site with one or more percutaneous pins or wires protruding from the skin, with one bandage having a narrower slit and the other bandage having a wider slit;

FIG. 10 schematically illustrates two bandage configurations designed to protect and provide antimicrobial activity at a surgical site with one or more percutaneous pins or wires protruding from the skin, with one being a single bandage and the other being a double bandage with one bandage being stacked over the other bandage, and a third bandage with a central opening but no slit for comparison;

FIG. 11 illustrates a surgical site with a hand that has been surgically repaired using two surgical pins or wires that protrude from the skin and bandages with central openings but no slits applied at the surgical site, with the percutaneous pins or wires extending through respective central openings of the bandages;

FIG. 12 illustrates a surgical site with a hand that has been surgically repaired using a surgical pin or wire that protrudes from the skin and a protective bandage according to the disclosure placed at the surgical site, with the percutaneous pin or wire extending through a slit of the bandage;

FIG. 13 illustrates a surgical site with a hand that has been surgically repaired using two surgical pins or wires that protrude from the skin and protective bandages according to the disclosure placed at the surgical site, with the percutaneous pins or wires extending through respective slits of the bandages;

FIG. 14 illustrates a surgical site with a foot that has been surgically repaired using a plurality of percutaneous surgical pins or wires that protrude from the skin and protective bandages according to the disclosure placed at the surgical with the percutaneous pins or wires extending through respective slits of the bandages; and

FIG. 15 illustrates a surgical site with a badly crushed finger that has been surgically repaired using a plurality of percutaneous surgical pins or wires that protrude from the skin and protective bandages according to the disclosure placed at the surgical with the percutaneous pins or wires extending through respective slits of the bandages.

FIG. 16 schematically illustrates a top perspective view of an embodiment of a unitary multi-aperture dressing body designed to protect the surgical site and provide an antimicrobial barrier at a surgical site having multiple percutaneous pins or wires.

FIG. 17 schematically illustrates a side cross-sectional view of an embodiment of a unitary multi-aperture dressing body formed as a single, continuous structure designed to protect the surgical site and provide an antimicrobial barrier at a surgical site having multiple percutaneous pins or wires.

FIGS. 18A, 18B, 18C, and 18D schematically illustrate a side cross-sectional view of a surgical site with a percutaneous pin or wire protruding from the skin and a multi-aperture dressing body applied at the surgical site, with the percutaneous pin or wire passing through the one aperture of the dressing body and a concentration gradient of antimicrobial agent that generally decreases with distance from the pin.

FIG. 18A schematically illustrates an embodiment in which the antimicrobial agent is impregnated in substantially all of the material comprising the unitary multi-aperture dressing body.

FIG. 18B schematically illustrates an embodiment in which the antimicrobial agent is impregnated in a layer of material that contacts both the pin and substantially all of the skin in contact with the bandage but does not comprise substantially all of the material comprising the unitary multi-aperture dressing body.

FIG. 18C schematically illustrates an embodiment in which the antimicrobial agent is impregnated in a layer of material that contacts the pin but does contact substantially all of the skin in contact with the bandage and does not comprise substantially all of the material comprising the unitary multi-aperture dressing body.

FIG. 18D schematically illustrates an embodiment in which the antimicrobial agent is impregnated in one of one or more regions of material that contact the pin but which do not contact substantially all of the skin in contact with the bandage and which do not comprise substantially all of the material comprising the unitary multi-aperture dressing body.

### DETAILED DESCRIPTION OF SOME EMBODIMENTS

The present disclosure relates to bandages (e.g., dressings) configured to provide antimicrobial protection, exudate absorption, and mechanical stability at a surgical site with one or more percutaneous pins or wires protruding beyond the skin. The bandage includes an adhesive layer, an absorbent layer, an antimicrobial agent adjacent to and/or impregnated in one or both of the absorbent layer and adhesive layer, and one or more slits through the bandage designed to accommodate and receive therethrough a percutaneous pin or wire. The bandages disclosed herein advantageously enhance and facilitate ease of bandaging and providing wound care at a surgical site with one or more percutaneous pins or wires protruding from the skin without disturbing the one or more percutaneous pins or wires.

As used herein, the term “percutaneous pin or wire” broadly refers to any percutaneous pin or wire extending through and out of the skin of a patient, such as surgical pins or wires, orthopedic pins or wires, pins and wires used in plastic surgery, and pins and wires used in ear nose and throat (ENT) procedures. In some embodiments, the “percutaneous pin or wire” may refer to a Kirschner wire (K-wire), external fixation pin, and/or a Steinmann pin.

As used herein, the term “wound care” generally refers to procedures commonly used by medical practitioners and the like to care for an open or surgical wound of a patient. For instance, wound care may include one or more of providing antimicrobial protection, absorbing wound exudate, or providing mechanical stability to one or more percutaneous pins or wires.

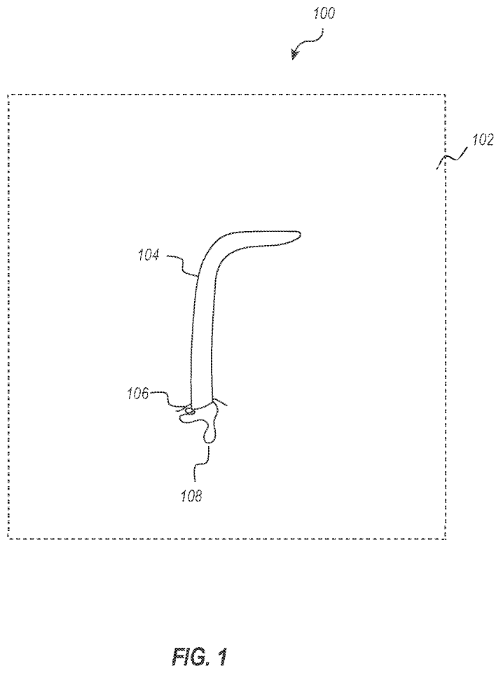

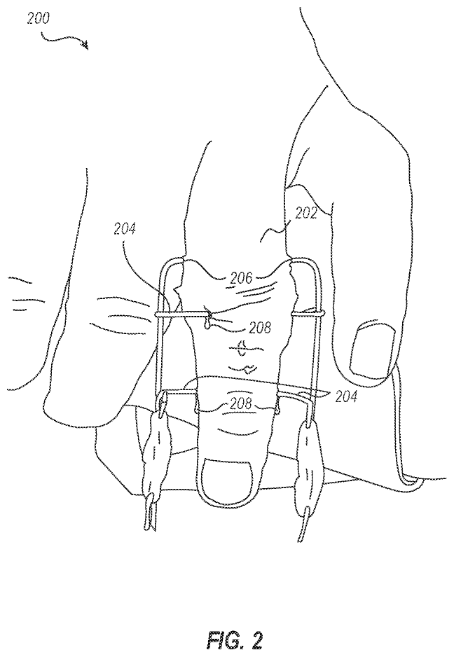

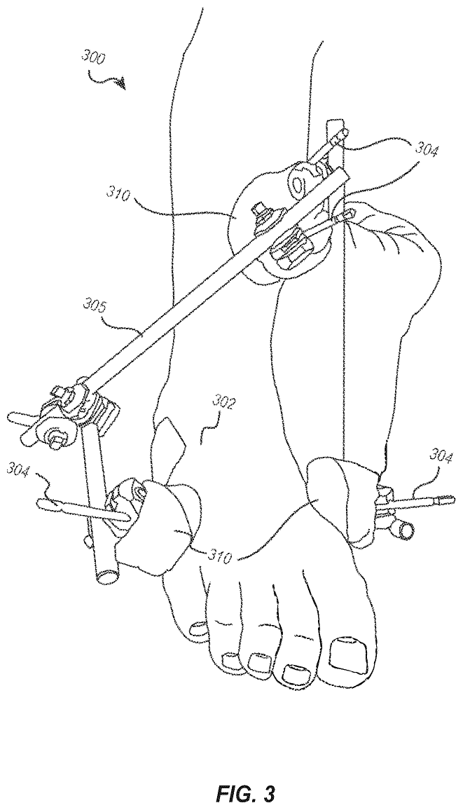

Surgical sites with one or more percutaneous pins or wires extending therefrom present a unique challenge to surgeons and other medical practitioners during initial placement, as well as medical practitioners and patients when it is desired to remove an old or dirty bandage and replace it with a fresh bandage during the recovery period following surgery. For instance, percutaneous pins or wires prevent closure of open wounds from which the percutaneous pins or wires extend until the percutaneous pins or wires have been removed (see FIGS. 1 and 2). Therefore, medical practitioners must keep such surgical sites bandaged to prevent excessive fluid leakage from the one or more open wounds and prevent infection or contamination of the one or more open wounds for as long as the percutaneous pins or wires remain at the surgical site. Currently, medical practitioners use makeshift bandages that are not designed for use with percutaneous (e.g., surgical or orthopedic) pins or wires (see FIG. 3).

Furthermore, percutaneous pins or wires are often bent, modified, or attached to external structures (e.g., as illustrated in FIGS. 1-3) to prevent excessive movement of the pins or wires, such as tearing of the skin or migration of the pins or wires beneath the skin of a patient. For example, percutaneous pins or wires may be bent into a Shepard's crook (illustrated in FIG. 1), may be interconnected together in a cage-like structure (illustrated in FIG. 2), or be fastened to external hardware (illustrated in FIG. 3).

Because surgical sites can have one or more percutaneous pins or wires that have been bent, modified, or attached to an external structure extending therefrom, traditional bandages or dressings used to cover and provide an antimicrobial barrier at a surgical site must be modified, sometimes extensively, in a manner that changes their intended use in order to accommodate percutaneous surgical pins or wires. Specifically, it is desirable for a bandage or dressing to adhere to and be flush with the skin surface at the surgical site to prevent fluid buildup and prevent contaminants and microbes from breaching the bandage and contaminating or infecting the surgical site.

Thus, traditional bandages must be cut, punctured, combined, secured with tape, or otherwise modified to meet the standard of care required to protect and provide a protective barrier to the surgical site with one or more percutaneous pins or wires extending therefrom. This modification process is not standardized and can be cumbersome or inconsistent, which can extend the amount of time open wounds at a surgical site are exposed to air as well as increase the risk that the percutaneous pins or wires will be disturbed when a modified bandage or dressing is placed and secured at the surgical site.

Furthermore, percutaneous pins or wires often remain in the body of a patient for extended periods of time (from weeks to months) to facilitate bone stabilization and healing. Therefore, patients are often required to periodically clean a surgical site with one or more percutaneous pins outside of a clinical setting. For example, patients may be instructed to clean the surgical site at home and change bandages at the surgical site that includes protruding pins or wires. Because patients typically lack the training or expertise to properly sterilize and dress an open wound, the bandages disclosed herein, which are specifically designed to standardize and simplify the process of providing an antimicrobial barrier and dressing an open wound with percutaneous pins or wires, greatly simplify and standardize the process, thus preventing errors by inexperienced patients or marginally competent medical practitioners.

The bandages disclosed herein overcome at least some of the aforementioned shortcomings of conventional bandages or dressings that must be modified to protect and provide an antimicrobial barrier at a surgical site with one or more percutaneous pins or wires extending therefrom. The disclosed bandages are specifically designed to protect and provide an antimicrobial barrier to surgical sites that include one or more percutaneous pins or wires. To facilitate ease of use by medical practitioners and patients, the disclosed bandages advantageously include an antimicrobial agent, an absorbent layer, an adhesive layer, and one or more slits designed to accommodate and receive therethrough, and circumferentially close or seal around, a percutaneous pin or wire.

The adhesive layer is advantageously configured to reliably adhere to tissue at the surgical site and prevent dislodgement of the bandage during its intended use. The adhesive layer facilitates easy attachment and removal of the bandage to and from the surgical site without further modification or application of tape. The adhesive layer is also advantageously configured so that the bandage can be removed from the surgical site without significantly damaging tissue. For instance, the adhesive layer may prevent discomfort and/or trauma to the surgical site as the bandage is removed from the surgical site. This is in contrast to conventional tape, which can adhere more strongly to skin and other tissue and cause damage thereto when removed, particularly when the skin or other tissue is still weak and tender following surgery.

The absorbent layer is advantageously a porous and/or hydrophilic substance configured to absorb wound exudate (e.g., blood, blood components, plasma, serum, and/or serosanguineous discharges) that may leak from one or more open wounds, such as those caused by the percutaneous (e.g., surgical and orthopedic) pins or wires. The absorbent layer is configured to absorb and contain wound exudate from, and facilitate healing of, the one or more open wounds. The absorbent layer reduces or minimizes further leakage from the one or more open wounds and increases the anti-infection properties of the bandage. Furthermore, the absorbent layer protects the skin from symptomatic pin or wire migration, thereby providing mechanical stabilization to the one or more percutaneous pins or wires.

The antimicrobial agent is advantageously provided within the absorbent layer, the adhesive layer, both the absorbent layer and the adhesive layer, and/or as separate antimicrobial layer (e.g., between the adhesive and absorbent layers), wherein the antimicrobial agent provides antimicrobial protection to a surgical site (e.g., disinfects and/or maintains sterility) when a bandage is placed thereon. The antimicrobial agent thus prevents complications related to infection of the one or more open wounds from which one or more percutaneous pins or wires extend.

The slit is a pre-cut opening across a portion of the width or diameter of the bandage and is tailored to receive and fit snugly around the percutaneous surgical pin or wire. When properly placed, the slit facilitates placement of the bandage circumferentially around the pin or wire in order to provide a reliable physical barrier to prevent ingress of contaminants at the surgical site, particularly at the wound provided by the protruding pin or wire. A properly placed bandage not only provides a reliable barrier to maintain sterility of the surgical site, it can also provide structural immobilization of the percutaneous pin or wire that minimizes movement of, or potential irritation caused by movement of, the percutaneous pin or wire. Furthermore, the slit facilitates placement of the bandage over and around a percutaneous pin or wire without disturbing it and without compromising the protective and antimicrobial properties of the bandage.

In some embodiments, the bandage further comprises a pre-cut central opening in communication with the slit, wherein the central opening is tailored to fit snugly around a percutaneous pin or wire, thereby facilitating proper placement of the bandage and potentially improving the seal around the pin or wire.

In some preferred embodiments, the materials used to form the bandage are flexible, allowing for the bandage to conform to the contours of surgical sites at any location on the body of a patient. The flexible materials can facilitate manipulation of the bandage in order to maximize adherence to and sealing of tissue and pin or wire. Furthermore, flexible materials can accommodate movement or shifting of the percutaneous pins or wires without dislodging or tearing.

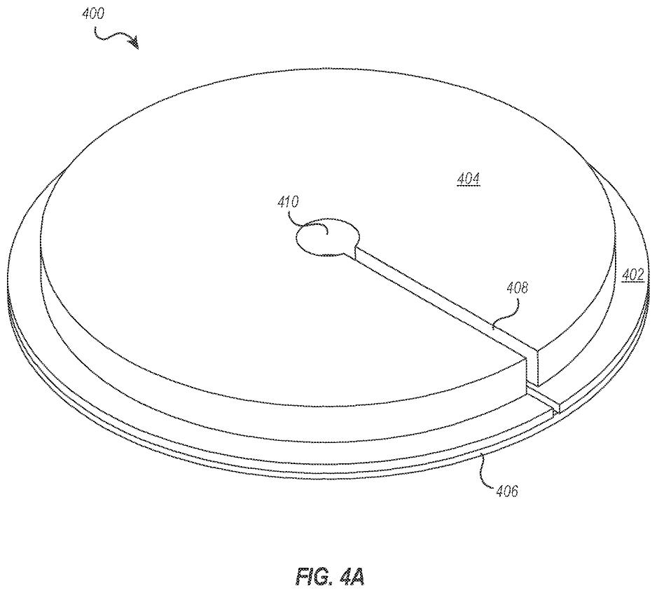

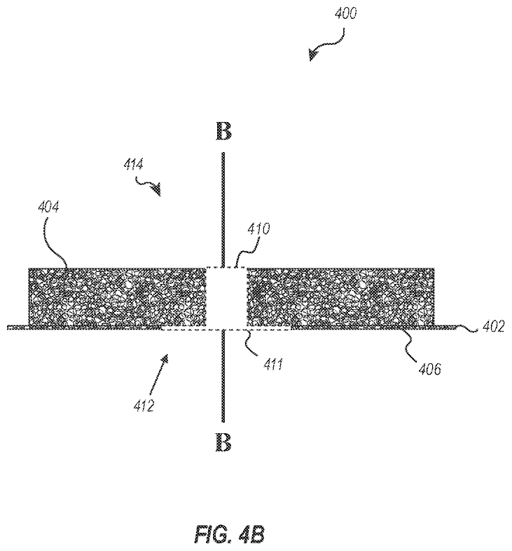

Turning now to the drawings, FIGS. 4A and 4B illustrate a first embodiment of a protective bandage 400 according to the disclosure for use in protecting and disinfecting to a surgical site with one or more percutaneous pins or wires. The bandage 400 includes an adhesive layer 402, which includes a first side configured to adhere to tissue and a second side opposite the first side, and an absorbent layer 404 adhered to the second side of the adhesive layer 402. A protective film or backing layer 406 initially covers the first side of the adhesive layer 402 to maintain the cleanliness and adhesive properties of the adhesive layer 402 during storage and prior to use. The protective film 406 can be removed from the adhesive layer 402 prior to use to expose the first side and permit it to adhere to tissue at a surgical site. As shown in FIG. 4B, the bandage 400 has a proximal side 412 oriented toward tissue during use and a distal side 414 opposite the proximal side 412.

The bandage 400 further includes a slit 408 extending partially across the width or diameter of the bandage 400 and a central opening 410 through the absorbent layer and an adhesive layer opening 411 through the adhesive layer. The slit 408 and central opening 410 facilitate placement of the bandage 400 at a surgical site and are configured to receive a surgical pin or wire (not shown) during placement of the bandage at a surgical site. The slit 408 permits the bandage 400 to be moved laterally relative to a surgical pin or wire protruding from the surgical site, so as to not disturb or put undue force on the percutaneous pin or wire. The central opening 410 is designed to surround and form a seal against an outer perimeter of the percutaneous pin or wire when the bandage 400 is placed at a surgical site. The central opening may be in the center of the bandage or may be offset depending on the length of the slit (e.g., it can be off center to match the length of the slit relative to the diameter or width of the bandage). In other embodiments, the central opening 410 can be omitted, with the flexibility of the bandage and absorbent layer being sufficient to conform to and seal around the percutaneous pin or wire.

The adhesive layer opening 411 through the adhesive layer 402 permits the absorbent layer 404 to contact the surgical site to facilitate absorption of wound exudate and provide antimicrobial activity (e.g., where the absorbent layer 404 includes an antimicrobial agent impregnated therein. Alternatively, in the case where a separate antimicrobial layer (not shown) is positioned between the adhesive layer 402 and the absorbent layer 404, the adhesive layer opening 411 permits direct contact of the antimicrobial layer to tissue at the surgical site. The adhesive layer opening 411 may have any desired size and can have a diameter that is about 10% to 20%, or about 90%, or about 20% to about 80%, or about 30% to about 40%, or 10% to 90%, or 20% to 3080%, or 30% to 40%, or 40% to 50%, or 50% to 60%, or 60% to 70%, or 70% to 80%, or 80% to 90% of the width or diameter of the bandage, or of any values, approximate values, ranges of values, or approximate ranges of values in any of the foregoing ranges. In some embodiments, the adhesive layer opening 411 has a diameter that substantially the same as the diametercentral opening 410, such that the adhesive layer adhesive layer covers substantially all of the skin-contacting surface 400.

As illustrated in FIG. 4B, the adhesive layer 402, absorbent layer 404, and backing layer 406 of the bandage 400 are axially aligned along a vertical axis B-B. As best seen in FIG. 4A, the slit 408 extends through the adhesive layer 402 and absorbent layer 404. The slit 408 can be formed by a first slit portion in the adhesive layer 402 and a second slit portion in the absorbent layer 404. The first slit and the second slit are advantageously axially aligned along vertical axis B-B such that they form the slit 408. It will be appreciated that the first and second slits can have the same or different widths depending on the intended use or functionality of the bandage 400. In some embodiments, an adhesive material may be disposed on at least a portion of the inside walls of slit 408 so that, when pressed together, slit 408 can be adhesively closed and form a physical barrier to microbes and dirt as if there were no slit 408.

The slit 408 is designed to accommodate a surgical wire or a percutaneous pin passing therethrough, wherein the protruding portion of the wire or pin may extend through the entirety of the bandage 400 via the slit 408. In various embodiments, the slit 408 can have a width that is the same as, similar to, or smaller than the diameter of the percutaneous pin or wire extending therethrough to reduce the amount of the surgical site exposed to open air. Accordingly, the slit 408 can have a width of about 0.4 mm to about 3.0 mm, or about 0.4 mm to about 5.0 mm, or about 0.4 mm to about 10.0 mm, or about 1.0 mm to about 3.0 mm, or about 1.0 mm to about 10.0 mm, or 0.4 mm to 3.0 mm, or 0.4 mm to 5.0 mm, or 0.4 mm to 10.0 mm, or 1.0 mm to 3.0 mm, or 1.0 mm to 10.0 mm, or of any values, approximate values, ranges of values, or approximate ranges of values in any of the foregoing ranges.

In some embodiments, the slit 408 may have a length that extends across at least a portion of the diameter (or width, depending on the shape) of the bandage 400. For instance, the slit may have a length that extends across about 10% to about 90%, or about 20% to about 50%, or about 10% to about 30%, or about 10% to about 15%, or 10% to 90%, or 20% to 50%, or 10% to 30%, or 10% to 15% of the width or diameter of the bandage, or of any values, approximate values, ranges of values, or approximate ranges of values in any of the foregoing ranges.

In some embodiments, a central opening 410 extends through the axially aligned layers of the bandage 400, wherein the central opening 410 extends through the proximal side 412 and the distal side 414 along a vertical axis B-B. As shown in FIG. 4B, the central opening 410 is formed in the absorbent layer 404, with a adhesive layer opening 411 formed in the adhesive layer 404. The central opening 410 and the adhesive layer opening 411 can be axially aligned along vertical axis B-B such that they form essentially concentric openings 410, 411. It will be appreciated that the openings 410, 411 can be non-concentric depending on the intended use or functionality of the bandage 400. The central opening 410 is in communication with the slit 408, such that the central opening 410 and slit 408 form a slit/opening complex configured to accommodate and fit snugly around a percutaneous pin or wire passing therethrough.

In embodiments that include the central opening 410, the central opening 410 is designed to accommodate a percutaneous wire or a surgical pin passing therethrough, wherein the percutaneous wire or pin may extend through the entirety of the bandage 400 via the central opening 410. In various embodiments, the central opening 410 can have a diameter that is the same as, similar to, or smaller than the diameter of the percutaneous wire or pin extending therethrough to reduce the amount of the surgical site exposed to open air. Accordingly, the central opening 410 can have a diameter in a range of about of about 0.4 mm to about 3.0 mm, or about 0.4 mm to about 5.0 mm, or about 0.4 mm to about 10.0 mm, or about 1.0 mm to about 3.0 mm, or about 1.0 mm to about 10.0 mm, or 0.4 mm to 3.0 mm, or 0.4 mm to 5.0 mm, or 0.4 mm to 10.0 mm, or 1.0 mm to 3.0 mm, or 1.0 mm to 10.0 mm, or of any values, approximate values, ranges of values, or approximate ranges of values in any of the foregoing ranges.

In some embodiments, as illustrated in FIG. 4B, the central opening 410 is positioned in the center of the bandage 400. However, it is appreciated that the central opening 410 may be off center, such as where it is desired for a portion of the bandage to extend further in one direction than another relative to the percutaneous pin or wire.

The adhesive layer 402, absorbent layer 404, and/or an antimicrobial layer (not pictured) can include (i.e., be impregnated with) an antimicrobial agent. Examples of antimicrobial agents include, but are not limited to, one or more of silver (e.g., silver nanoparticles and silver salts, such as silver nitrate and silver sulfadiazine, ionic silver, and nanocrystalline silver), iodine (e.g., povidone iodine and cadexomer iodine), chlorhexidine (e.g., chlorhexidine gluconate), polyhexamethylene biguanide (PHMB), honey, antibiotics (e.g., gentamicin, mupirocin, and bacitracin), hypochlorous acid, gentian violet, and essential oils (e.g., tea tree oil), and/or other substances capable of providing an antimicrobial barrier or other antimicrobial activity to a surgical site or wound.

The absorbent layer 404 can be formed from a hydrophilic material capable of absorbing fluid leakage from a wound site. Examples of hydrophilic materials include, but are not limited to, silicone, cotton wool, gauze, lint, plasters, foam, polyurethane foam, alginate, hydrocolloid, hydrogel, and or others capable of absorbing fluid leakage from a surgical or other wound site. In some embodiments, the absorbent layer 402 may be impregnated with a hemostatic agent, examples of which include one or more of thrombin, thrombin enzyme, prothrombin complex concentrate, dried plasma, cyanoacrylate, fibrin sealant, tranexamic acid, microfibrillar collagen, microporous polysaccharide spheres, gelatin matrix, oxidized regenerated cellulose, alginate, albumin, glutaraldehyde, alum (hydrated salts of aluminum and alkali metals) aluminum chloride, zinc chloride, ferric sulfates, and/or others capable of reducing bleeding or other fluid leakage from a surgical or other wound site.

The adhesive layer 402 forms the proximal side 412 of the bandage 400, wherein the adhesive layer 402 is configured to contact tissue at the surgical site. In some instances, the absorbent layer 404 may extend through one or more portions of the adhesive layer 402 (e.g., through adhesive layer opening 411) such that part of the absorbent layer 404 may be exposed at the distal side 414 to improve the absorbent and/or antimicrobial properties of the bandage 400. For instance, the absorbent layer 404 may cover 10% to about 90%, or about 20% to about 50%, or about 10% to about 30%, or about 10% to about 15%, or about 0%, or 0%, or 10% to 90%, or 20% to 50%, or 10% to 30%, or 10% to 15% of the surface area of the distal side 414, or of any values, approximate values, ranges of values, or approximate ranges of values in any of the foregoing ranges.

The adhesive layer 402 can be formed from or include an adhesive material configured to attach bandage 400 to tissue at a surgical site, examples of which include, but are not limited to, hydrocolloids, acrylic polymer films, cyanoacrylate films, silicone films, and/or other materials capable of attaching to skin at a surgical site. In some embodiments, the adhesive layer 402 is formed from a gel-like or malleable substance (e.g., hydrocolloid) such that the adhesive layer 402 can make good contact with and adhere to tissue around a percutaneous pin or wire passing through the slit 408 and/or central opening 410. In some embodiments, the adhesive layer 402 includes a protective film 406 (e.g., backing layer) that may be removed from the adhesive layer 402 prior to use. The protective film 406 maintains the cleanliness and adhesive properties of the adhesive layer 402 during storage and prior to use.

In some embodiments, the adhesive layer 402 can be impregnated with an antimicrobial agent, such that the adhesive layer 402 provides easy attachment of bandage 400 to tissue as well as provide antimicrobial protection to a wound.

The individual layers and/or combined layers form the bandage 400, wherein the bandage 400 has a height or cross-sectional thickness taken along the vertical axis B-B in a range of about 1 mm to about 20 mm, or about 1 mm to about 50 mm, or about 15 mm to about 25 mm, or about 45 mm to about 55 mm, or 1 mm to 20 mm, or 1 mm to 50 mm, or 15 mm to 25 mm, or 45 mm to 55 mm, or of any values, approximate values, ranges of values, or approximate ranges of values in any of the foregoing ranges. The adhesive layer 402 may have a relatively smaller cross-sectional thickness, such as about 1 mm to about 10 mm, or about 5 mm to about 10 mm, or about 1 mm to about 25 mm, or about 10 mm to about 15 mm, or about 20 mm to about 25 mm, or 1 mm to 10 mm, or 5 mm to 10 mm, or 1 mm to 25 mm, or 10 mm to 15 mm, or 20 mm to 25 mm, or of any values, approximate values, ranges of values, or approximate ranges of values in any of the foregoing ranges, such that the majority of the height or cross-sectional thickness of the bandage 400 comprises or is provided by the absorbent layer 404. Furthermore, the bandage 400 may have a diameter or width in a range from about 5 mm to about 50 mm, or about 1 mm to about 50 mm, or about 45 mm to about 55 mm, 5 mm to 50 mm, or 1 mm to 50 mm, or 45 mm to 55 mm, or of any values, approximate values, ranges of values, or approximate ranges of values in any of the foregoing ranges. The foregoing ranges are applicable to the other embodiments disclosed herein.

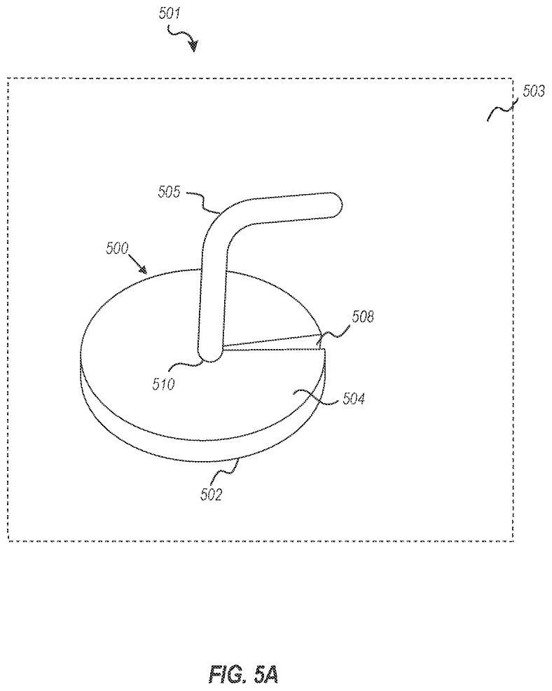

FIGS. 5A and 5B illustrate a bandage 500 applied to a surgical site 501, wherein the percutaneous pin/wire 505 is received through a slot 508 and central opening 510. The adhesive layer 502 is placed on the skin surface 503, attaching the bandage 500 to the surgical site 501 without modification, which is a substantial improvement over makeshift or ad hoc dressings currently used, such as wrapping with gauze, securing with tape, or other methods of securing a bandage to a surgical site. As illustrated in FIGS. 5A and 5B, the bandage 500 provides a barrier between the wound (covered by bandage 500), which is formed by the percutaneous pin or wire 505 penetrating through and protruding from the skin 503, and open air, provides antimicrobial protection by the antimicrobial agent, and absorbs wound exudate (covered by bandage 500) by the absorbent layer 504.

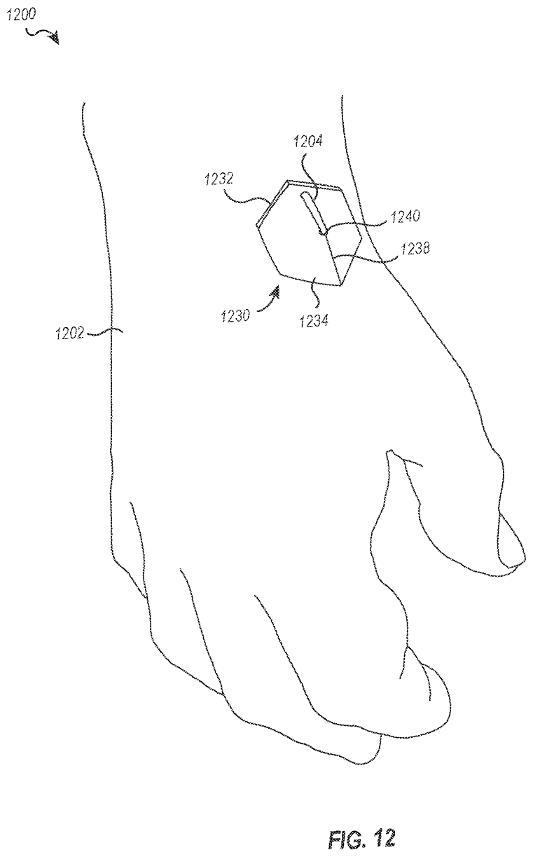

The bandage 500 and other bandages disclosed herein can be used to improve wound care, increase the ease of bandaging various surgical sites, such as those illustrated in FIGS. 1-3 and further illustrated in FIGS. 12-16, and improve patient outcomes by standardizing the process of bandaging a surgical site with one or more percutaneous pins or wires.

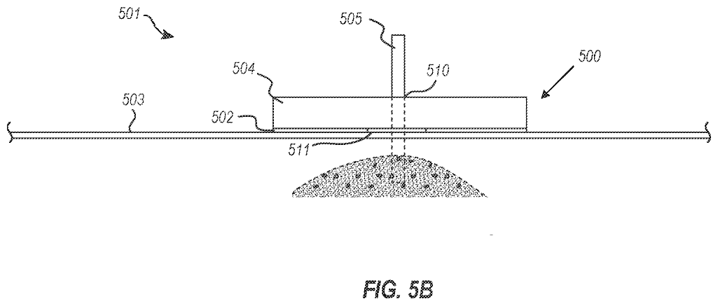

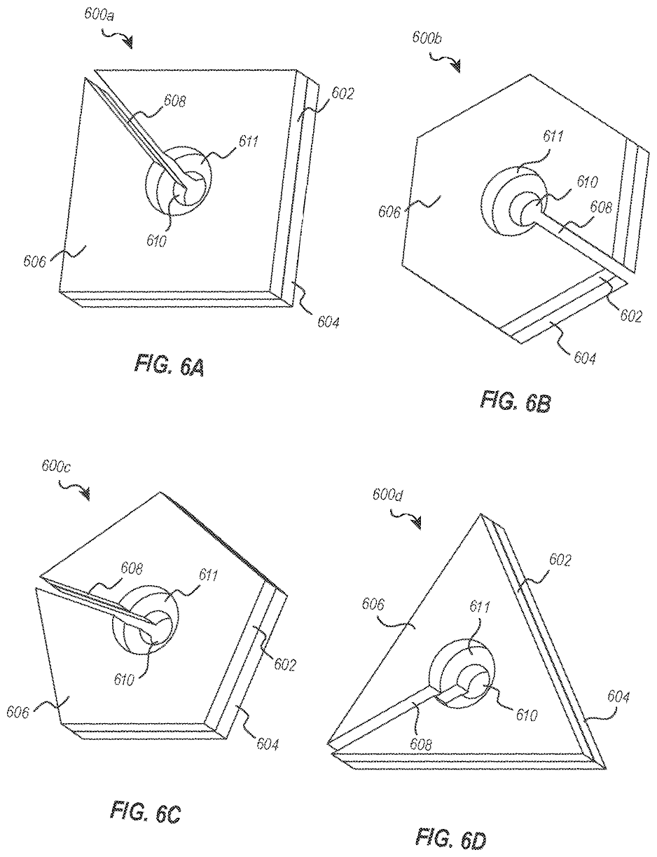

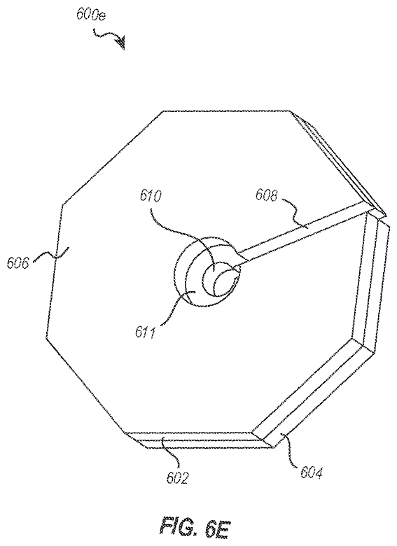

In some embodiments, bandages disclosed herein may have a desired geometric shape. For instance, bandages may be circular, as illustrated in FIGS. 4A-4B and 5A-5B. Alternatively, such as those illustrated in FIGS. 6A-6E, bandages 600 may be square, rectangular, hexagonal, pentagonal, triangular, octagonal, or another geometric shape that allows the application of multiple adjacent bandages 600 to attach to and protect a surgical site with one or more percutaneous pins or wires extending therefrom. The shape of the bandage 600 may assist a medical practitioner in covering or substantially covering a skin surface at a surgical site comprising more than one percutaneous pin or wire to form a continuous and uninterrupted barrier to prevent contamination and infection. The bandages 600 illustrated in FIGS. 6A-6E are shown bottom side up, with the backing layer 606 at the top.

FIG. 6A illustrates a square-shaped bandage 600a having an adhesive layer 602, an absorbent layer 604, a backing layer on the proximal side of adhesive layer 602, a slit 608 passing partially through the bandage 600a, a pin or wire opening 610 through the absorbent layer 604, and a larger opening 611 through the adhesive layer 602 so that a portion of the absorbent layer 604 and associated antimicrobial agent can contact skin at a surgical site. Although the slit 608 is shown extending from a corner of the square-shaped bandage 600a to the opening 610, it will be appreciated that it may extend from a side of the bandage 600a to the opening 610.

FIG. 6B illustrates a hexagonal-shaped bandage 600b having an adhesive layer 602, an absorbent layer 604, a backing layer on the proximal side of adhesive layer 602, a slit 608 passing partially through the bandage 600b, a pin or wire opening 610 through the absorbent layer 604, and a larger opening 611 through the adhesive layer 602 so that a portion of the absorbent layer 604 and associated antimicrobial agent can contact skin at a surgical site. Although the slit 608 is shown extending from a corner of the hexagonal-shaped bandage 600b to the opening 610, it will be appreciated that it may extend from a side of the bandage 600b to the opening 610.

FIG. 6C illustrates a pentagonal-shaped bandage 600c having an adhesive layer 602, an absorbent layer 604, a backing layer on the proximal side of adhesive layer 602, a slit 608 passing partially through the bandage 600c, a pin or wire opening 610 through the absorbent layer 604, and a larger opening 611 through the adhesive layer 602 so that a portion of the absorbent layer 604 and associated antimicrobial agent can contact skin at a surgical site. Although the slit 608 is shown extending from a corner of the pentagonal-shaped bandage 600c to the opening 610, it will be appreciated that it may extend from a side of the bandage 600c to the opening 610.

FIG. 6D illustrates a triangular bandage 600d having an adhesive layer 602, an absorbent layer 604, a backing layer on the proximal side of adhesive layer 602, a slit 608 passing partially through the bandage 600d, a pin or wire opening 610 through the absorbent layer 604, and a larger opening 611 through the adhesive layer 602 so that a portion of the absorbent layer 604 and associated antimicrobial agent can contact skin at a surgical site. Although the slit 608 is shown extending from a corner of the triangular bandage 600d to the opening 610, it will be appreciated that it may extend from a side of the 600d to the opening 610.

FIG. 6E illustrates an octagonal-shaped bandage 600e having an adhesive layer 602, an absorbent layer 604, a backing layer on the proximal side of adhesive layer 602, a slit 608 passing partially through the bandage 600e, a pin or wire opening 610 through the absorbent layer 604, and a larger opening 611 through the adhesive layer 602 so that a portion of the absorbent layer 604 and associated antimicrobial agent can contact skin at a surgical site. Although the slit 608 is shown extending from a corner of the octagonal-shaped bandage 600e to the opening 610, it will be appreciated that it may extend from a side of the bandage 600e to the opening 610.

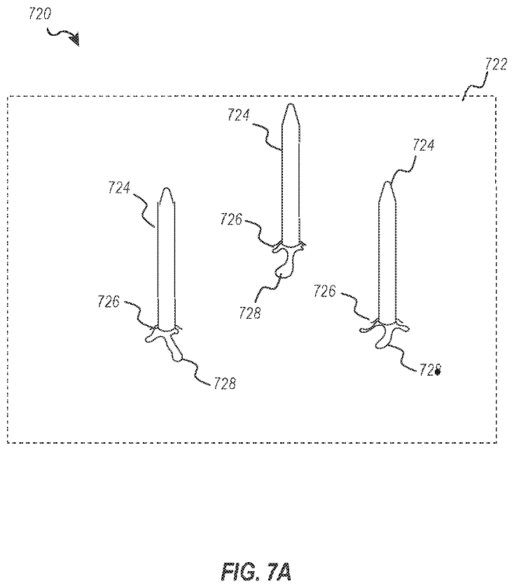

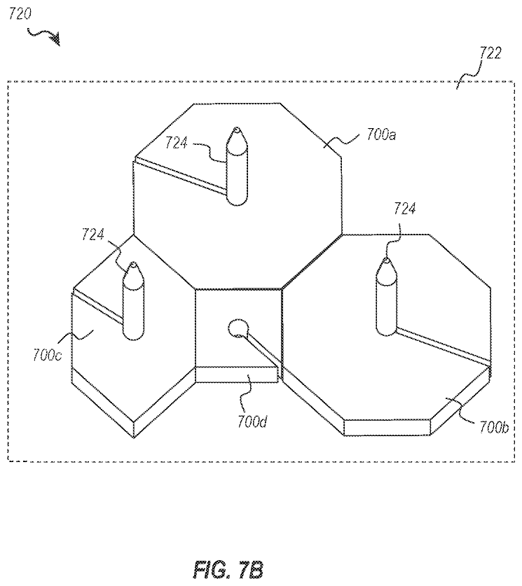

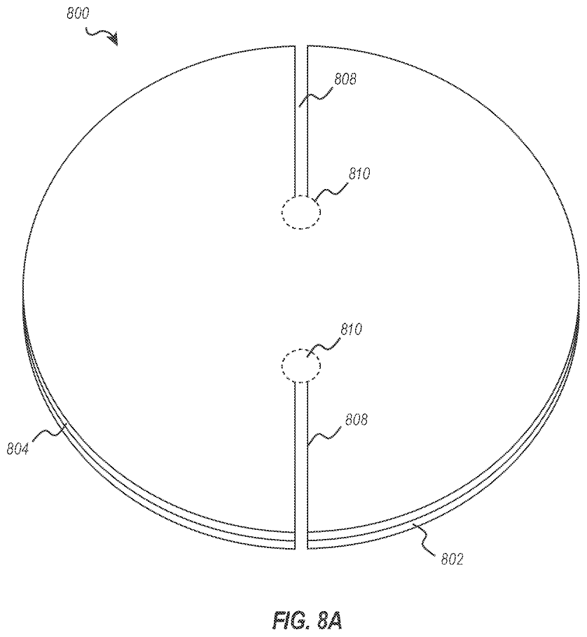

By way of example, FIGS. 7A and 7B illustrate how multiple bandages 700 of various geometric shapes and sizes can be combined to form a bandaging system to more fully cover, protect, and provide antimicrobial activity at a large region of a surgical site. FIG. 7A illustrates a surgical site 720 comprising skin surface 722, wherein three percutaneous pins/wires 724 extend therefrom, leaving wounds 726 exposed at the skin surface 722 and permitting leakage of fluid or exudate 728 from the wounds 726.

FIG. 7B illustrates an arrangement of multiple bandages 700 disposed at the surgical site 720. The multiple bandages 700 comprise different shapes that can be selected to fit together and form a bandage system that comprehensively covers the surgical site 720 without significant gaps and/or overlap. As illustrated, the bandage system includes two octagonal-shaped bandages 700a, 700b that abut each other, a hexagonal-shaped bandage 700c that abuts bandage 700a, and a square-shaped bandage 700d that abuts each of bandages 700a, 700b, 700c to fill in a gap between bandages 700b, 700c. As illustrated, one or more bandages 700 can be used to cover a portion of the skin surface 722 without being placed around a percutaneous pin or wire 724 (i.e., the squares-shaped bandage 700d).

In some instances, a patient may be instructed to clean and re-dress their surgical recovery site with one or more percutaneous pins or wires outside of a clinical setting. In such instances, patients without medical training would be expected to re-dress such a surgical recovery site aseptically and without disturbing the one or more percutaneous pins or wires. A bandage or a kit of bandages as disclosed herein may be prescribed to a patient in order to facilitate easy unbandaging and rebandaging of the recovery site. A kit of bandages may be particularly useful when a patient is required to replace bandages at a recovery site with a plurality of percutaneous pins or wires. For example, a kit may provide a patient with multiple sets of correctly-sized and shaped bandages used to comprehensively cover the recovery site and accommodate the percutaneous wires or pins. The kit may additionally include wound cleaning supplies, instructions for correct application of the bandages, and/or other information regarding at-home care.

By way of example, a kit may comprise one or more sets of the bandages as illustrated in FIG. 7B, instructions on where to place each bandage at the surgical recovery site, and instructions on how often to change the bandages. The instructions may include written instructions and/or a schematic of the wound site (such as the schematic illustration presented in FIG. 7B).

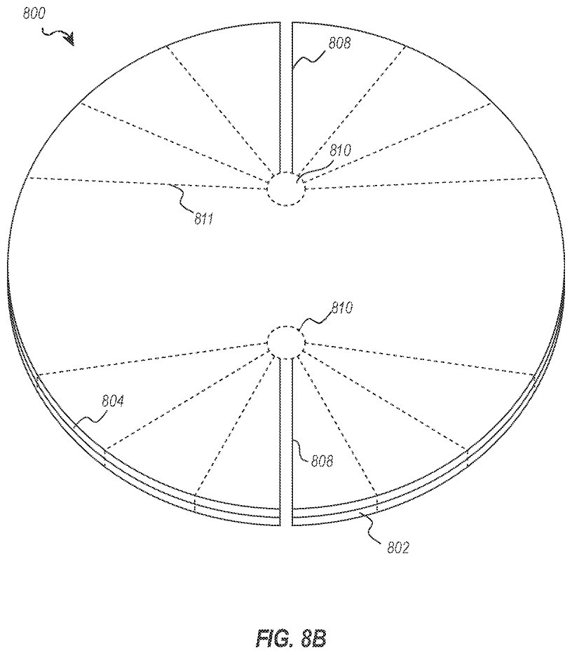

FIG. 8A illustrates another embodiment of a bandage 800, which comprises an adhesive layer 802, an absorbent layer 804, and a plurality (e.g., two) of slits 808 to accommodate and receive therethrough a plurality of (e.g., two) percutaneous pins or wires extending from a surgical site. The plurality of slits 808 are formed through an axis across the diameter of the bandage 800 so as to be axially aligned. Alternatively, the slits 808 may have other orientations to permit different angles of percutaneous pin or wire insertion. Each slit 808 can be used to accommodate one or more percutaneous pins or surgical wires using a single bandage. The bandage 800 may optionally include pin or wire openings 810, which are illustrated as dotted lines in FIG. 8A, in communication with respective slits 808. In some instances, each slit 808 of the plurality of slits 808 may include an adhesive disposed on a portion of the inside walls of each slit 808, wherein if pressed together, the walls can be adhesively connected to form a continuous barrier.

FIG. 8B illustrates another embodiment of a bandage 800, which comprises an adhesive layer 802, an absorbent layer 804, and a plurality (e.g., two) of slits 808 to accommodate and receive therethrough a plurality (e.g., two) of percutaneous pins or wires extending from a surgical site. The plurality of slits 808 are formed through an axis across the diameter of the bandage 800 so as to be axially aligned. Alternatively, the slits 808 may have other orientations to permit different angles of pin or wire insertion. Each slit 808 can be used to accommodate one or more percutaneous pins or surgical wires using a single bandage. The bandage 800 may optionally include pin or wire openings 810, which are illustrated as dotted lines in FIG. 8B, in communication with respective slits 808. Furthermore, bandage 800 may include one or more perforations 811 that extend through the thickness of the bandage 800, such that the perforations 811 allow a medical practitioner to open one or more additional slits into the bandage 800.

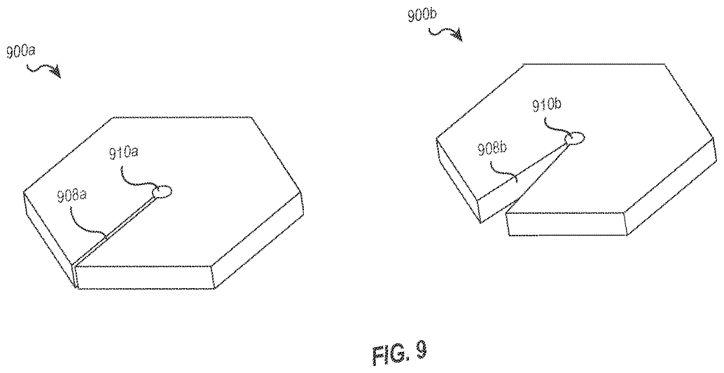

FIG. 9 illustrates two bandages 900 with different slit designs to protect and sterilize a surgical site with one or more percutaneous pins or wires protruding from the skin, with one bandage having a narrower slit and the other bandage having a wider slit. A first bandage 900a includes a relatively narrow slit 908a with essentially parallel side walls and a central opening 910a. A second bandage 900b includes a wider slit 908b with diverging side walls and a central opening 910b, which can be the same or different diameter as central opening 910a. An advantage of first bandage 900a is the slit is narrow and easy to close together in order to cover and protect skin and wound at a surgical site with one or more percutaneous pins or wires. An advantage of second bandage 900b is the slit is wider at its opening making it easier to slide a percutaneous pin or wire through. The second bandage can be flexible to permit a practitioner to manually close the slit when placing the bandage against the skin. Alternatively, the slit may naturally close together if the bandage is placed in a concave region at a surgical site.

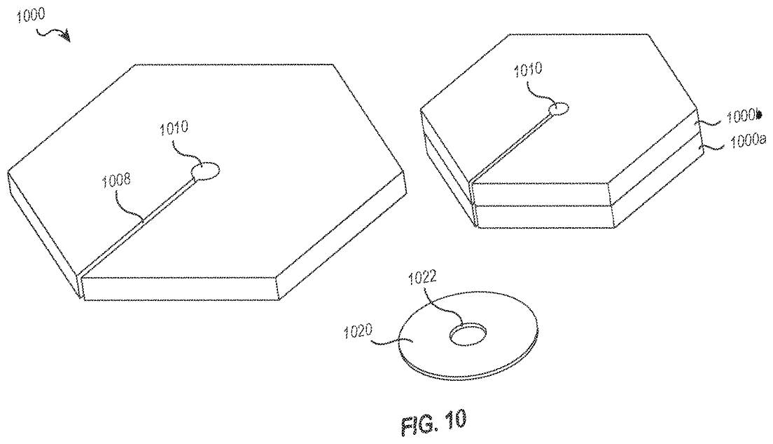

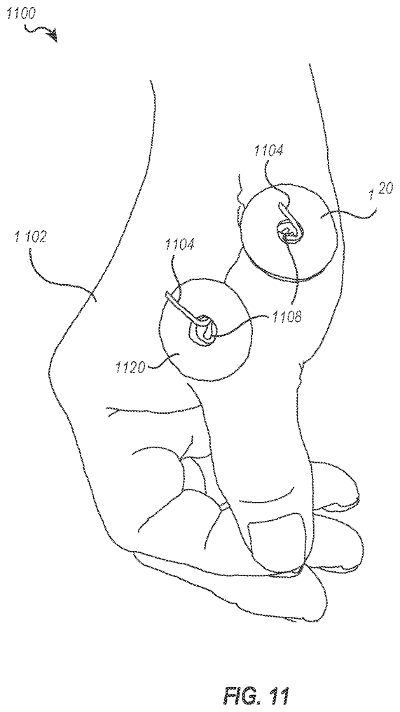

FIG. 10 schematically illustrates two bandage configurations designed to protect and provide antimicrobial activity at a surgical site with one or more percutaneous pins or wires protruding from the skin, and a third bandage with a central opening but no slit for comparison. A first bandage configuration comprises a single bandage 1000 having a slit 1008 and wire or pin opening 1010. A second bandage configuration comprises a first bandage 1000a for application to skin at a surgical site and a second bandage 1000b placed over the first bandage 1000a, each having a slit 1008 and wire or pin opening 1010 which are advantageously aligned. The second bandage configuration can be constructed before placement at the surgical site, or it may be constructed by placing the first bandage 1000a at the surgical site and then placing the second bandage 1000a over the first bandage 1000a.

The third bandage 1020 is a conventional dressing for placement of a catheter therethrough. The third bandage 1020 has a central hole 1022 that is sized to loosely accommodate the diameter of a catheter. However, third bandage 1020 has no adhesive layer or backing layer and is not able to adhere to skin or other tissue in order to stabilize and protect a surgical site with one or more percutaneous pins or wires protruding from the skin. The third bandage 1020 is also of relatively small diameter since its only function is to receive a catheter therethrough attached to a port, not protect the skin at a surgical site.

FIG. 11 illustrates a surgical site 1100 with a hand that has been surgically repaired using two percutaneous pins or wires 1104 that protrude from the skin 1102. A pair of bandages 1120 with central holes but no slits or adhesive are applied at the surgical site of the hand, with the percutaneous pins or wires 1104 extending through respective central holes of the bandages 1120. Because the bandages 1120 lack an adhesive layer, they are unable to adhere to the skin 1102 at the surgical site and provide a reliable antimicrobial barrier. In addition, because the central holes are much larger than the pins or wires 1104, the bandages 1120 are unable to absorb and stop the flow of exudate 1108 and provide a reliable antimicrobial barrier.

FIG. 12 illustrates a surgical site 1200 with a hand that has been surgically repaired using a percutaneous surgical pin or wire 1204 that protrudes from the skin 1202. A protective bandage 1230 according to the disclosure is placed at the surgical site, with the pin or wire 1204 extending through a slit 1338 of the bandage 1230. The bandage 1230 includes an adhesive layer 1232 that reliably adheres the bandage 1230 to the skin 1202 and an absorbent layer 1234 that includes an antimicrobial agent and is configured to absorb any exudate (not shown) that may flow from the wound at the interface of the skin 1202 and wire or pin 1204.

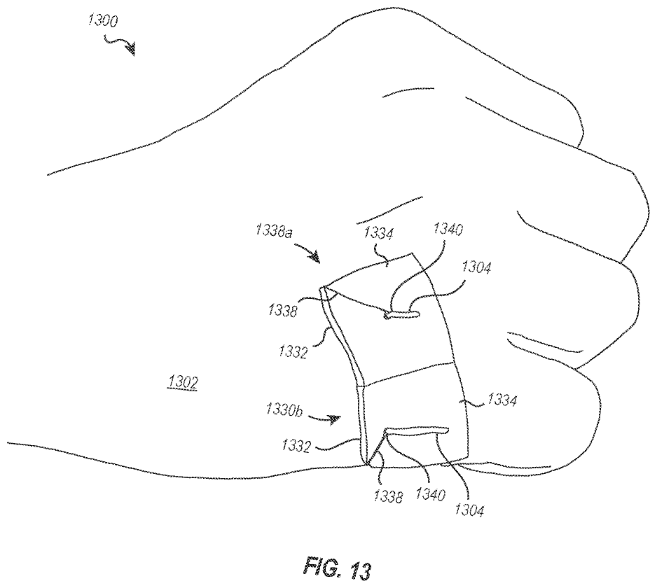

FIG. 13 illustrates a surgical site 1300 with a hand that has been surgically repaired using two percutaneous pins or wires 1304 that protrude from the skin 1302. Two square-shaped protective bandages 1330a, 1330b according to the disclosure are placed adjacent to each other at the surgical site, with the pins or wires 1304 extending through respective slits 1338 of the bandages 1330a, 1330b. The bandages 1330a, 1330b each include an adhesive layer 1332 that reliably adheres the bandages 1330a, 1330b to the skin 1302 and an absorbent layer 1334 that includes an antimicrobial agent and is configured to absorb any exudate (not shown) that may flow from the wound at the interface of the skin 1302 and wires or pins 1304. Abutment of the protective bandages 1330a, 1330b provides a continuous protective layer at the surgical with no gaps therebetween.

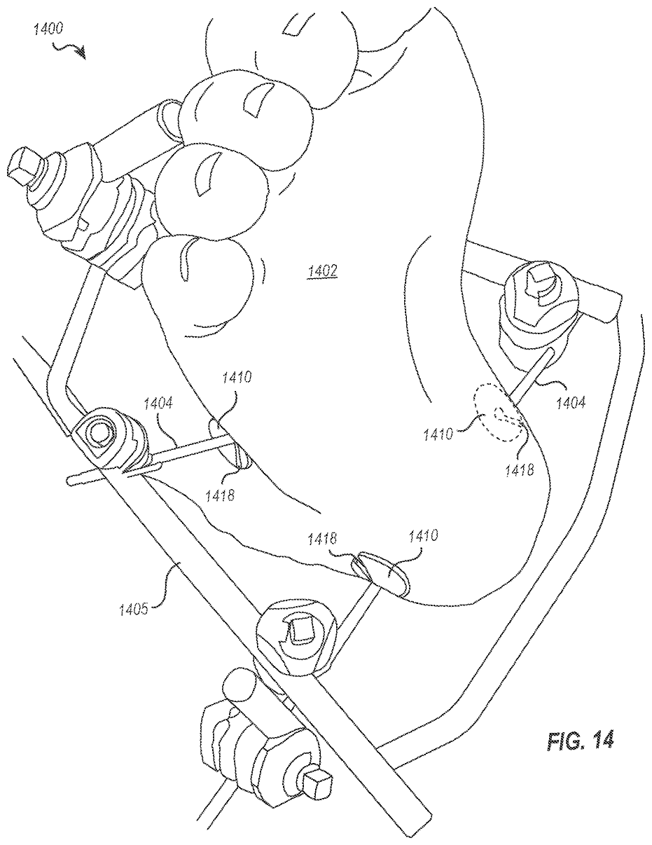

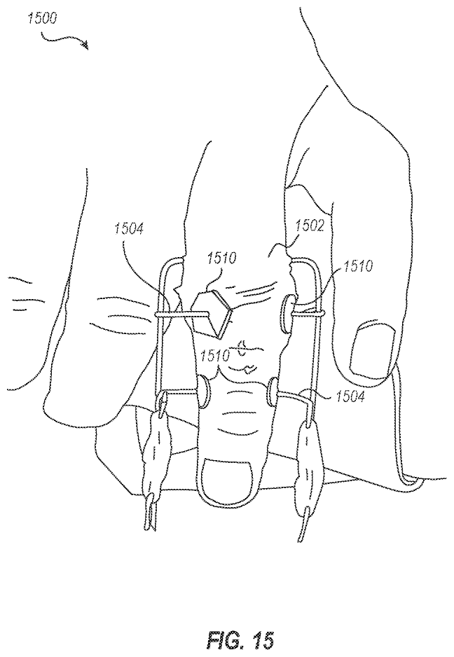

FIG. 14 illustrates a surgical site 1400 with a foot that has been surgically repaired using a plurality of percutaneous pins or wires 1404 that protrude from the skin 1402. The pins or wires 1404 are attached to an external stabilization structure 1405, which can complicate placement of bandages as discussed above relative to FIG. 3. Protective bandages 1410 according to the disclosure are placed at the surgical site 1400 with the pins or wires 1404 extending through respective slits 1408 of the bandages 1410. The bandages 1410 each include an adhesive layer that reliably adheres the bandages 1410 to the skin 1402 and an absorbent layer that includes an antimicrobial agent and is configured to absorb any exudate (not shown) that may flow from the wound at the interface of the skin 1402 and wires or pins 1404.

For the sake of comparison, FIG. 14 shows how bandages 1410 according to the disclosure are much smaller and provide a bandage system that is much simpler to apply and replace compared to the bulky makeshift gauze wrap 310 dressings used in FIG. 3, which shows the same surgically repaired foot, percutaneous pins, and external stabilization structure.

FIG. 15 illustrates a surgical site 1500 with a badly damaged finger that has been surgically repaired using a plurality of percutaneous pins or wires 1504 that protrude from the skin 1502. The percutaneous pins or wires 1504 are interconnected in a cage-like structure, which can complicate placement of bandages as discussed above relative to FIG. 2. Protective bandages 1510 according to the disclosure are placed at the surgical site 1500 with the percutaneous pins or wires 1504 extending through respective slits of the bandages 1510. The bandages 1510 each include an adhesive layer that reliably adheres the bandages 1510 to the skin 1502 and an absorbent layer that includes an antimicrobial agent and is configured to absorb any exudate (not shown) that may flow from the wound at the interface of the skin 1502 and the percutaneous wires or pins 1504.

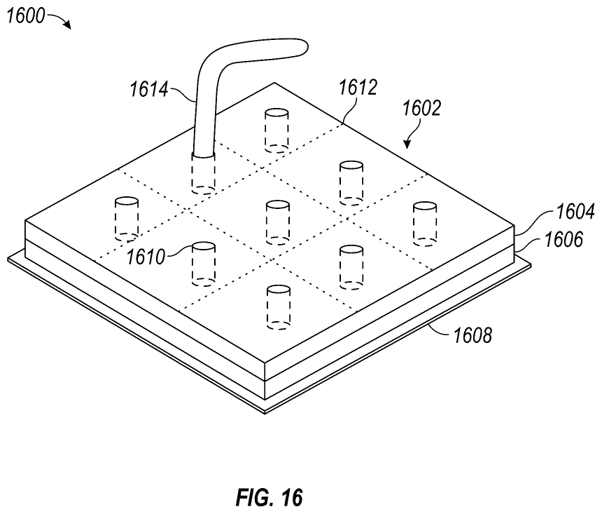

FIG. 16 schematically illustrates a bandage 1600 comprising a unitary (i.e., single piece) multi-aperture dressing body 1602 configured to receive and interface with at least one percutaneous pin or wire 1614. The unitary dressing body 1602 comprises an antimicrobial layer 1604, an absorbent layer 1606, and an adhesive layer 1608 arranged in a layered construction. In some embodiments, the antimicrobial layer 1604, the absorbent layer 1606, and the adhesive layer 1608 are initially provided as respective sheets, assembled in a stacked relationship, and bonded to one another.

The unitary dressing body 1602 defines at least one aperture 1610 configured to receive the at least one percutaneous pin or wire 1614. The aperture 1610 extends through the antimicrobial layer 1604 and through the absorbent layer 1606 such that interior surfaces of the aperture 1610 expose cross-sectional portions of both layers to the surface of the percutaneous pin 1614.

In some embodiments, the absorbent layer 1606 and the antimicrobial layer 1604 are discrete layers. In other embodiments, the antimicrobial layer 1604 and the absorbent layer 1606 can at least partially overlap or can be substantially coextensive, such that a single material region provides both absorptive functionality and antimicrobial functionality.

FIG. 16 further illustrates at least one perforation 1612 associated with the unitary dressing body 1602. In some embodiments, the perforation 1612 comprises a prefabricated perforated region arranged to define a tearable zone. A clinician may then tear or cut along the perforation 1612 to change the shape of the bandage 1600 to adapt the shape of the bandage 1600 to a specific patient geometry.

While FIG. 16 illustrates a bandage 1600 that is substantially square in shape, other shapes may be provided, such as circular, hexagonal, pentagonal, or other polygonal or continuous shapes. Further, while FIG. 16 illustrates an arrangement of perforations 1612 that are substantially grid-like in nature such that they subdivide the bandage 1600 into regions that are substantially square, other arrangements of perforations may be provided, such that the regions defined by the perforations are circular, hexagonal, pentagonal, or otherwise polygonal or represented by continuous shapes.

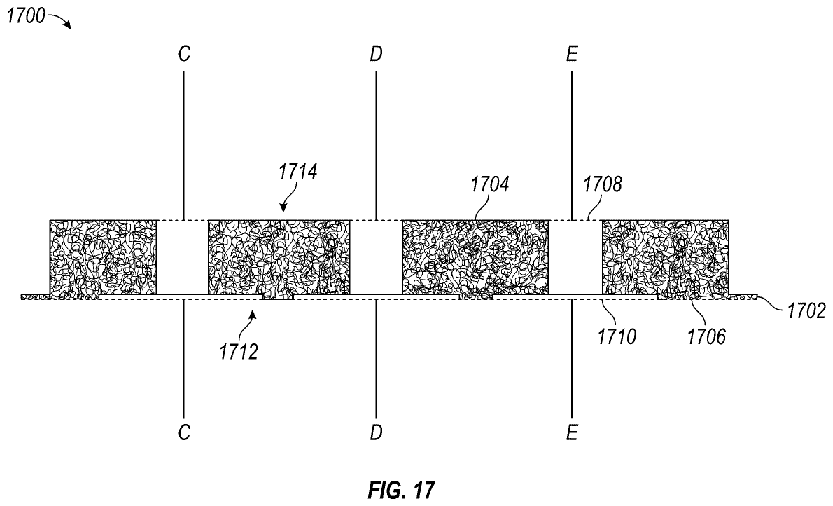

FIG. 17 schematically illustrates a cross-sectional view of a bandage 1700 having a layered construction extending between a proximal side 1712 and a distal side 1714. The bandage 1700 comprises an adhesive layer 1702, a combination antimicrobial and absorbent layer 1704, and a protective film or backing layer 1706 arranged in a stacked relationship. In some embodiments, not pictured, the antimicrobial and absorbent layers may be two separate layers bonded together. The adhesive layer 1702 is configured to attach the bandage 1700 to tissue at a surgical site. The combination layer 1704 comprises a material configured provide absorbent functionality and antimicrobial functionality.

In some embodiments, the adhesive layer 1702 includes a protective film or backing layer 1706 that may be removed from the adhesive layer 1702 prior to use. The protective film 1706 maintains the cleanliness and adhesive properties of the adhesive layer 1702 during storage and prior to use.

Some embodiments of the bandage 1700 include at least one aperture 1708 and a larger opening 1710 extending through the layered structure. The adhesive layer 1702, combination layer 1704, and backing layer 1706 are vertically aligned such that each aperture 1708 extends axially along a vertical axis. For example, the aperture may be axially aligned with vertical axis C-C, D-D, or E-E, or another axis as defined by another aperture. A percutaneous pin or wire (not pictured) can extend through the aperture 1708 or the larger opening 1710 when the bandage 1700 is applied at one or more pin sites. In some embodiments, the opening geometry is selected or modified to provide clearance for the percutaneous member while maintaining surrounding contact between the combination layer 1704 and tissue adjacent the pin site. In some embodiments, the combination layer 1704 is positioned directly adjacent the percutaneous member to vertically wick exudate away from the pin site and to maintain a relatively clean and dry interface.

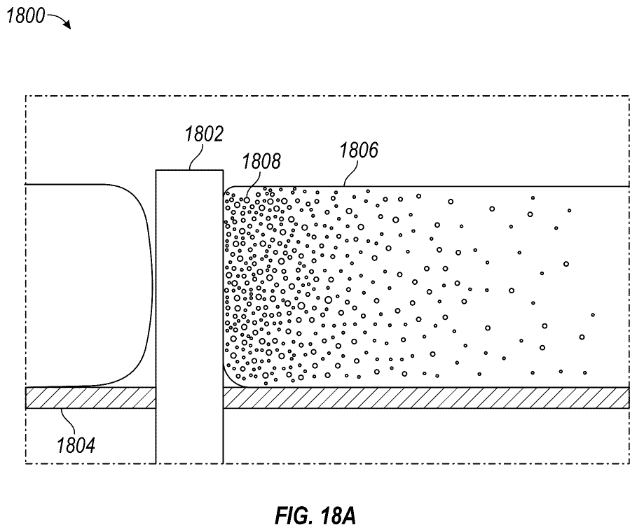

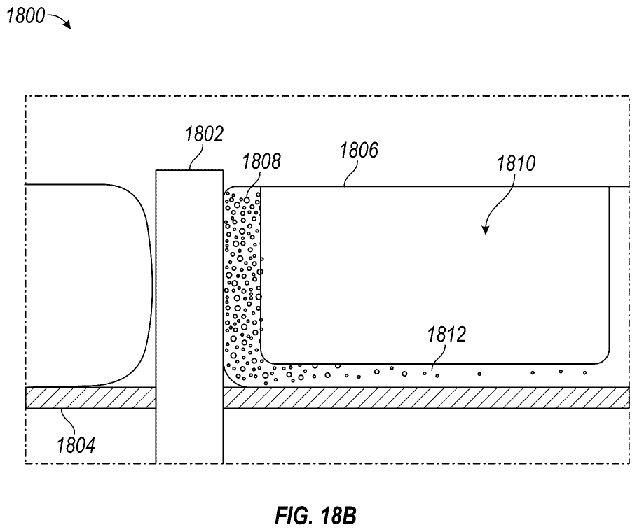

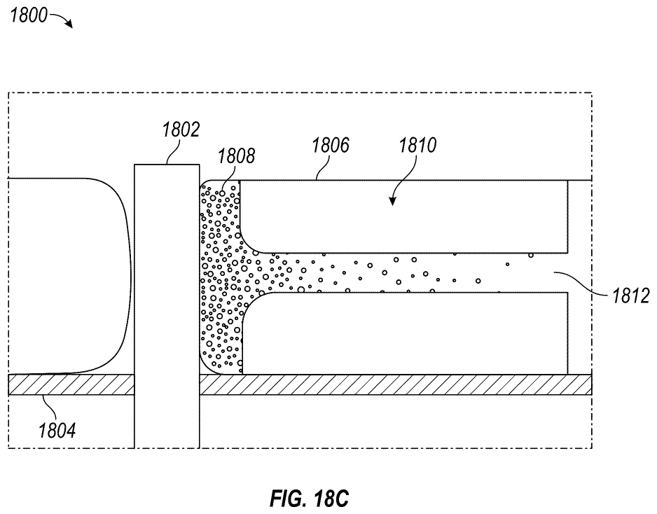

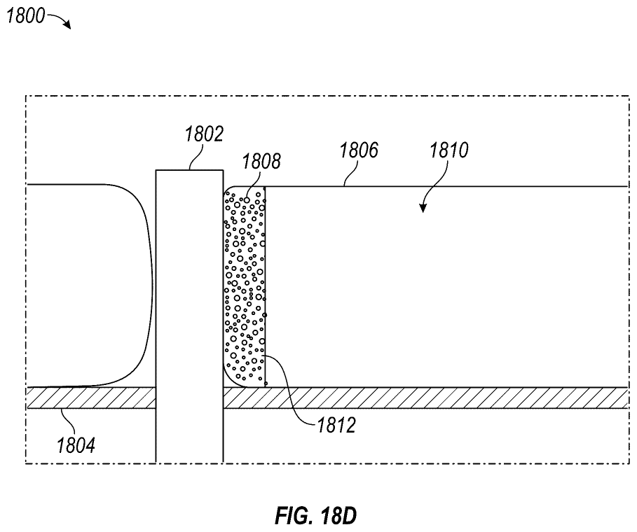

FIG. 18A schematically illustrates a bandage 1800 positioned relative to a percutaneous pin 1802 extending through skin 1804. The bandage 1800 includes a combination antimicrobial and absorbent layer 1806 configured to provide both fluid-management functionality and antimicrobial functionality within a common material body. An antimicrobial agent 1808 is distributed within the combination layer 1806 in a spatially varying manner such that the concentration or loading of the antimicrobial agent 1808 is greater in a region immediately adjacent the percutaneous pin 1802 and decreases with increasing distance from the percutaneous pin 1802. The progressively reduced presence of the antimicrobial agent 1808 in regions farther from the percutaneous pin 1802 can reduce unnecessary antimicrobial exposure in portions of the bandage 1800 that overlie intact skin while preserving localized antimicrobial performance where infection risk is highest.

FIG. 18B schematically illustrates a bandage 1800 positioned relative to a percutaneous pin 1802 extending through skin 1804, wherein a combination antimicrobial and absorbent layer 1806 includes an absorbent region 1810 and an antimicrobial region 1812. In the illustrated embodiment, the antimicrobial region 1812 is localized adjacent the percutaneous pin 1802 and along a skin-facing portion of the bandage 1800, while the absorbent region 1810 occupies the remainder of the combination layer 1806. In some embodiments, the absorbent region 1810 and the antimicrobial region 1812 are integrally formed within a laminated or otherwise unitary dressing construction.

FIG. 18C schematically illustrates a bandage 1800 positioned relative to a percutaneous pin 1802 extending through skin 1804, wherein a combination layer 1806 includes one or more absorbent regions 1810 and an antimicrobial region 1812 disposed therebetween. The antimicrobial region 1812 is localized adjacent the percutaneous pin 1802 and extends into the interior of the combination layer 1806, while absorbent regions 1810 are positioned on opposite sides of the antimicrobial region 1812.

FIG. 18D schematically illustrates a bandage 1800 positioned relative to a percutaneous pin 1802 extending through skin 1804, wherein a combination layer 1806 includes an absorbent region 1810 and a localized antimicrobial region 1812 positioned immediately adjacent the percutaneous pin 1802 near the pin-skin interface. In the illustrated embodiment, the antimicrobial region 1812 contains an antimicrobial agent 1808 and is confined to a region of the bandage 1800 coaxial with the percutaneous pin 1802, while the absorbent region 1810 occupies the remaining portion of the combination layer 1806. This arrangement can provide concentrated antimicrobial activity where the percutaneous pin 1802 emerges from the skin 1804 while minimizing antimicrobial material in portions of the bandage 1800 spaced farther from the pin site.

Although FIGS. 18A-18D each illustrate an embodiment in which the bandage 1800 includes a single aperture associated with a single percutaneous pin 1802, this is merely illustrative. Further, although FIGS. 18A-18D illustrate certain architectures on only one side of the pin for clarity, the illustrated architectures may extend circumferentially around the pin, including around the full 360 degrees of the pin. The omission of corresponding features or reference numerals on the opposite side of the pin in FIGS. 18A-18D is for drawing clarity and should not be understood as limiting the illustrated architecture to only one side of the pin. It should be understood that the arrangements described with respect to FIGS. 18A-18D can likewise be implemented in a unitary dressing body having a plurality of apertures configured to receive a corresponding plurality of percutaneous pins. In such embodiments, each aperture may be associated with any of the antimicrobial and absorbent architectures described herein, including for example an antimicrobial region wherein concentration or loading of the antimicrobial agent is greater in a region immediately adjacent the percutaneous pin, an antimicrobial region localized adjacent the percutaneous pin and along a skin-facing portion of the bandage, one or more absorbent regions and an antimicrobial region disposed therebetween, an antimicrobial region of the bandage that is coaxial with the percutaneous pin, or any combination thereof, such that the unitary multi-aperture dressing body provides coordinated treatment around multiple pin sites within a single integrated bandage structure.

A method for protecting and providing antimicrobial activity (e.g., provide an antimicrobial barrier) at a surgical site with one or more percutaneous pins or wires involves providing a bandage comprising an adhesive layer, an absorbent layer, and an antimicrobial agent forming a separate layer and/or impregnated within one or both of the adhesive layer and absorbent layer, wherein one or more slits extend through the adhesive layer and the absorbent layer. For example, one or more of the various embodiments of a bandage, as described hereinabove, may be provided. The method further involves removing a protective film (i.e., a backing layer) from a proximal face of the adhesive layer and placing the adhesive layer so that the proximal face contacts the surgical site and so that each of the one or more percutaneous pins or wires extends through a respective one of the one or more slits. In some instances, more than one bandage may be used. Placing the adhesive layer at the surgical site causes the bandage to become attached to the surgical site, allowing the absorbent layer of the bandage to absorb fluids that may leak out of wounds at the surgical site, and allowing the antimicrobial agent to provide antimicrobial protection to the surgical site.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

While certain examples have been described, these examples have been presented by way of example only and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

The terms “approximately,” “about,” and “substantially” as used herein represent an amount close to the stated amount that still performs a desired function or achieves a desired result. For example, in some examples, as the context may dictate, the terms “approximately,” “about,” and “substantially,” may refer to an amount that is within less than or equal to 10% of the stated amount. The term “generally” as used herein represents a value, amount, or characteristic that predominantly includes or tends toward a particular value, amount, or characteristic. As an example, in certain examples, as the context may dictate, the term “generally parallel” can refer to something that departs from exactly parallel by less than or equal to 20 degrees. All ranges are inclusive of endpoints.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results, unless provided otherwise herein. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Although the foregoing invention has been described in terms of certain preferred embodiments, other embodiments will be apparent to those of ordinary skill in the art. Additionally, other combinations, omissions, substitutions, and modifications will be apparent to the skilled artisan, in view of the disclosure herein. Accordingly, the present invention is not intended to be limited by the recitation of the preferred embodiments but is instead to be defined by reference to the appended claims.

The terminology used in the description presented herein is not intended to be interpreted in any limited or restrictive manner and unless otherwise indicated refers to the ordinary meaning as would be understood by one of ordinary skill in the art in view of the specification. Furthermore, embodiments may comprise, consist of, consist essentially of, several novel features, no single one of which is solely responsible for its desirable attributes or is believed to be essential to practicing the embodiments herein described. As used herein, the section headings are for organizational purposes only and are not to be construed as limiting the described subject matter in any way. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein unless provided otherwise herein.

Although this disclosure is in the context of certain embodiments and examples, those of ordinary skill in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of ordinary skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

What is claimed is:

1. A surgical dressing for a surgical site having a plurality of percutaneous pins or wires protruding through skin, the surgical dressing comprising:
a unitary dressing body;
an adhesive layer configured to attach the unitary dressing body to tissue at the surgical site;
an absorbent region;
an antimicrobial region; and
a plurality of tearable perforated regions configured to selectively be torn to form a plurality of apertures, the plurality of apertures configured to receive the plurality of percutaneous pins or wires,
wherein the surgical dressing is configured for placement over the surgical site to prevent microbial infection of the surgical site.

2. The surgical dressing of claim 1, wherein at least one of the plurality of perforated regions comprises a perforation pattern comprising a slit, a circle, a keyhole shape, and/or a multi-segment compliant opening.

3. The surgical dressing of claim 1, wherein one or more of the plurality of perforations are arranged in a pre-defined grid pattern.

4. The surgical dressing of claim 3, wherein the pre-defined grid pattern is a square grid pattern, a hexagonal grid pattern, a pentagonal grid pattern, or a radial pattern.

5. The surgical dressing of claim 3, wherein the one or more perforations comprise perforations separated by intervening land portions having a width of 0.3 mm to 2 mm.

6. The surgical dressing of claim 1, further comprising an adhesive material disposed along an interior surface of at least one of the plurality of apertures.

7. The surgical dressing of claim 1, wherein the surgical dressing is configured to attenuate transmission of motion from at least one of the plurality of percutaneous pins or wires to the surgical site.

8. The surgical dressing of claim 1, wherein the surgical dressing further comprises one or more scores extending partially through one or more layers of the unitary dressing body.

9. The surgical dressing of claim 1, wherein the antimicrobial region and the absorbent region comprise discrete layers adhered to one another within the unitary dressing body.

10. The surgical dressing of claim 1, wherein the antimicrobial region and the absorbent region are at least partially overlapping or substantially coextensive within the unitary dressing body.

11. The surgical dressing of claim 1, wherein the antimicrobial region has a greater concentration of antimicrobial agent near a respective aperture than in a region spaced farther from the aperture.

12. The surgical dressing of claim 1, wherein the unitary dressing body comprises a visual indicator configured to provide a visible indication that a predetermined condition of at least a portion of the surgical dressing has reached a predetermined state.

13. The surgical dressing of claim 12, wherein the visual indicator is configured to provide the visible indication by changing color.

14. The surgical dressing of claim 12, wherein the visual indicator is configured to provide the visible indication by changing in opacity.

15. The surgical dressing of claim 12, wherein the predetermined condition is time in use, a depletion of an antimicrobial agent in the antimicrobial material, or an accumulation of exudate.

16. The surgical dressing of claim 12, wherein the visual indicator is configured to provide the visible indication in response to a level of contamination exceeding a predetermined value.

17. The surgical dressing of claim 12, wherein the visual indicator is configured to provide the visible indication when a pH exceeds about 8.0 or when a moisture level exceeds about 50-75%.

18. The surgical dressing of claim 12, wherein the visual indicator is configured to provide the visible indication when a concentration of an antimicrobial agent is below about 0.1% to about 2.0%.

19. The surgical dressing of claim 1, further comprising a supporting backing layer disposed over a surface of the unitary dressing body opposite a skin-contacting surface of the surgical dressing.

20. The surgical dressing of claim 1, wherein the antimicrobial region includes an antimicrobial agent that comprises polyhexamethylene biguanide or chlorhexidine gluconate.

21. The surgical dressing of claim 1, wherein the antimicrobial region comprises polyurethane foam.

22. The surgical dressing of claim 1, wherein the adhesive layer extends across substantially all of a skin-contacting surface of the surgical dressing.

23. The surgical dressing of claim 1, wherein the aperture diameter is about 3 mm to about 6 mm.

24. The surgical dressing of claim 1, wherein the thickness of the surgical dressing is about 15 mm to about 25 mm.

25. The surgical dressing of claim 1, wherein the absorbent region comprises a first absorbent material positioned closer to a respective percutaneous pin or wire and a second absorbent material positioned farther from the respective percutaneous pin or wire, the second absorbent material having a greater absorbency than the first absorbent material to promote transfer of moisture or exudate away from the respective percutaneous pin or wire.
